SEQUENCE LISTING (18 sequences)

SEQ ID NO: 1 - RNA, 19 nt, artificial
  siRNA targeting luciferase
cuuacgcugaguacuucga

SEQ ID NO: 2 - RNA, 19 nt, artificial
  siRNA targeting BRCA1
guggguguuggacagugua

SEQ ID NO: 3 - RNA, 19 nt, artificial
  siRNA targeting BRCA1
gaaggagcuuucaucauuc

SEQ ID NO: 4 - RNA, 19 nt, artificial
  siRNA targeting CDK12
gagacuagacaaugagaaa

SEQ ID NO: 5 - RNA, 19 nt, artificial
  siRNA targeting CDK12
gcugaauaacagugggcaa

SEQ ID NO: 6 - RNA, 19 nt, artificial
  siRNA targeting BRCA2
gacucuaggucaagauuua

SEQ ID NO: 7 - RNA, 19 nt, artificial
  siRNA targeting BRCA2
gaagaaugcagguuuaaua

SEQ ID NO: 8 - RNA, 18 nt, artificial
  siRNA targeting RAD51
gggauuugugaagccaaa

SEQ ID NO: 9 - RNA, 19 nt, artificial
  siRNA targeting RAD51
ccaacgaugugaagaaauu

SEQ ID NO: 10 - DNA, 20 bp, artificial
  synthetic oligonucleotide primer
gccaaggcaagatctagagg

SEQ ID NO: 11 - DNA, 18 bp, artificial
  synthetic oligonucleotide primer
gttgccaacacgagctga

SEQ ID NO: 12 - DNA, 20 bp, artificial
  synthetic oligonucleotide primer
tgatcatggatgcgctcaag

SEQ ID NO: 13 - DNA, 20 bp, artificial
  synthetic oligonucleotide primer
ttgcgagatcacctcaaacc

SEQ ID NO: 14 - DNA, 20 bp, artificial
  synthetic oligonucleotide primer
gaaggtgaaggtcggagtca

SEQ ID NO: 15 - DNA, 20 bp, artificial
  synthetic oligonucleotide primer
aatgaaggggtcattgatgg

SEQ ID NO: 16 - DNA, 20 bp, artificial
  synthetic oligonucleotide primer
cactgcctgtctctgaccaa

SEQ ID NO: 17 - DNA, 20 bp, artificial
  synthetic oligonucleotide primer
caggagaacagggctgaaag

SEQ ID NO: 18 - protein, 6 aa, artificial
  FLAG tag
DYKDDK

(19) United States
(12) Patent Application Publication
(10) Pub. No.: US 2021/0386750 A1
(43) Pub. Date: Dec. 16, 2021

(54) METHODS AND MATERIALS FOR TREATING CANCER

(71) Applicant: Mayo Foundation for Medical Education and Research, Rochester, MN (US)

(72) Inventors: Larry M. Karnitz, Rochester, MN (US); Arun Kanakkanthara, Rochester, MN (US)

(21) Appl. No.: 17/285,001

(22) PCT Filed: Oct. 24, 2019
(86) PCT No.: PCT/US2019/057836

§ 371 Date: Apr. 13, 2021

Related U.S. Application Data
(60) Provisional application No. 62/863,065, filed on Jun. 18, 2019.
(60) Provisional application No. 62/751,358, filed on Oct. 26, 2018.

(51) Int. Cl. A61K 31/53; A61K 31/506; A61K 31/65; A61K 33/243; A61K 31/235; A61P 35/00

(52) U.S. Cl. CPC A61K 31/53; A61K 31/506; A61P 35/00; A61K 33/243; A61K 31/235; A61K 31/65

(57) ABSTRACT

This document provides methods and materials involved in treating mammals having cancer. For example, a mammal having a breast cancer 1 (BRCA1)-deficient cancer and/or a nicotinamide N-methyltransferase (NNMT) overexpressing cancer can be treated by administering one or more agents that can inhibit mitochondrial metabolism (e.g., one or more oxidative phosphorylation (OXPHOS) inhibitors and/or one or more inhibitors of a mitochondrial polypeptide) and/or one or more agents that can inhibit glucose transport (e.g., one or more inhibitors of a glucose transporter polypeptide such as glucose transporter 1 (GLUT1)) to the mammal. In some cases, one or more OXPHOS inhibitors can be administered to a mammal having a BRCA1-deficient cancer and/or a NNMT overexpressing cancer in combination with one or more poly(ADP-ribose) polymerase (PARP) inhibitors and/or one or more platinum compounds.

### CROSS-REFERENCE TO RELATED APPLICATIONS

This application claims the benefit of U.S. Patent Application Ser. No. 62/751,358, filed on Oct. 26, 2018, and claims the benefit of U.S. Patent Application Ser. No. 62/863,065, filed on Jun. 18, 2019. The disclosures of the prior applications are considered part of (and is incorporated by reference in) the disclosure of this application.

### STATEMENT REGARDING FEDERAL FUNDING

This invention was made with government support under CA194498 and CA136393 awarded by the National Institutes of Health. The government has certain rights in the invention.

### BACKGROUND

### 1. Technical Field

This document relates to methods and materials involved in treating mammals having cancer. For example, a mammal having a breast cancer 1 (BRCA1)-deficient cancer, a cyclin-dependent kinase 12 (CDK12)-deficient cancer, and/or a nicotinamide N-methyltransferase (NNMT) overexpressing cancer can be treated by administering one or more agents that can inhibit mitochondrial metabolism (e.g., one or more oxidative phosphorylation (OXPHOS) inhibitors and/or one or more inhibitors of a mitochondrial polypeptide) and/or one or more agents that can inhibit glucose transport (e.g., one or more inhibitors of a glucose transporter polypeptide such as glucose transporter 1 (GLUT1)) to the mammal. In some cases, one or more OXPHOS inhibitors can be administered to a mammal having a BRCA1-deficient cancer, a CDK12-deficient cancer, and/or a NNMT overexpressing cancer either alone or in combination with one or more poly(ADP-ribose) polymerase (PARP) inhibitors, one or more platinum compounds, and/or one or more chemotherapy agents that induce DNA crosslinks.

### 2. Background Information

Altered metabolism is a hallmark of cancer that has garnered much interest as a therapeutic target. One agent that targets tumor metabolism is VLX600, an iron chelator in Phase I clinical trials (see, e.g., clinicaltrials.gov/ct2/show/NCT02222363?term=vlx-600). Despite the fact that VLX600 is in clinical trials, it remains unclear how to select/identify tumors that are most likely to respond to this agent.

### SUMMARY

This document provides methods and materials involved or treating mammals in need thereof (e.g., mammals having cancer). For example, a mammal having cancer (e.g., a BRCA1-deficient cancer, a CDK12-deficient cancer, and/or a NNMT overexpressing cancer) can be treated by administering one or more OXPHOS inhibitors to the mammal. In some cases, one or more OXPHOS inhibitors can be used in combination with one or more PARP inhibitors, one or more platinum compounds, and/or one or more chemotherapy agents that crosslink DNA to treat a mammal having cancer (e.g., a BRCA1-deficient cancer, a CDK12-deficient cancer, and/or a NNMT overexpressing cancer). In some cases, a mammal can be identified as having a BRCA1-deficient cancer, based at least in part, on the cancer having one or more cancer cells having one or more modifications (e.g., loss-of-function modifications) in one or more nucleic acid sequences that encode a polypeptide associated with homologous recombination (HR). In some cases, a mammal can be identified as having a NNMT overexpressing cancer, based at least in part, on the cancer having one or more cancer cells that express an elevated level of a NNMT polypeptide.

As demonstrated herein, VLX600 disrupted HR. Also as demonstrated herein, BRCA1 depletion in ovarian cancer cell lines sensitized them to OXPHOS inhibitors VLX600 and ceritinib. In addition, VLX600 used in combination with cisplatin or in combination with the PARP inhibitor olaparib was more cytotoxic to BRCA1-deficient ovarian cancer cells than any agent alone.

Having the ability to identify a particular cancer treatment that a mammal (e.g., a human) is most likely to respond allows clinicians to provide an individualized approach in selected cancer treatments.

In general, one aspect of this document features methods for treating a mammal having cancer where the methods can include, or consist essentially of, (a) identifying a mammal as having a BRCA1-deficient cancer, and (b) administering an OXPHOS inhibitor to the mammal to increase the susceptibility of said cancer to treatment with a platinum compound or a PARP inhibitor. The mammal can be a human. The BRCA1-deficient cancer can be selected from the group consisting of ovarian cancers, breast cancers, pancreatic cancers, prostate cancers, skin cancers, renal cancers, liver cancers, stomach cancers, colon cancers, colorectal cancers, bladder cancers, and oral squamous cell cancers. The BRCA1-deficient cancer can be an ovarian cancer. The OXPHOS inhibitor can be selected from the group consisting of VLX600 and ceritinib. The OXPHOS inhibitor can be VLX600. The method also can include administering the platinum compound to the mammal. The platinum compound can be cisplatin. The method also can include administering the PARP inhibitor to the mammal. The PARP inhibitor can be olaparib.

In another aspect, this document features methods for treating a mammal having cancer where the methods can include, or consist essentially of, administering an OXPHOS inhibitor to a mammal identified as having a BRCA1-deficient cancer under conditions where the susceptibility of the cancer to treatment with a platinum compound or a PARP inhibitor increases. The mammal can be a human. The BRCA1-deficient cancer can be selected from the group consisting of ovarian cancers, breast cancers, pancreatic cancers, prostate cancers, skin cancers, renal cancers, liver cancers, stomach cancers, colon cancers, colorectal cancers, bladder cancers, and oral squamous cell cancers. The BRCA1-deficient cancer can be an ovarian cancer. The OXPHOS inhibitor can be selected from the group consisting of VLX600 and ceritinib. The OXPHOS inhibitor can be VLX600. The method also can include administering the platinum compound to the mammal. The platinum compound can be cisplatin. The method also can include administering the PARP inhibitor to the mammal. The PARP inhibitor can be olaparib.

In another aspect, this document features methods for treating a mammal having cancer where the methods can include, or consist essentially of, (a) identifying the mammal as having a BRCA1-deficient cancer, and (b) administering an inhibitor of a mitochondrial polypeptide to the mammal to increase the susceptibility of the cancer to treatment with a platinum compound or a PARP inhibitor. The mammal can be a human. The BRCA1-deficient cancer can be selected from the group consisting of ovarian cancers, breast cancers, pancreatic cancers, prostate cancers, skin cancers, renal cancers, liver cancers, stomach cancers, colon cancers, colorectal cancers, bladder cancers, and oral squamous cell cancers. The BRCA1-deficient cancer can be an ovarian cancer. The BRCA1-deficient cancer can be a breast cancer. The inhibitor of a mitochondrial polypeptide can be tigecycline. The method also can include administering the platinum compound to the mammal. The platinum compound can be cisplatin. The method also can include administering the PARP inhibitor to the mammal. The PARP inhibitor can be olaparib.

In another aspect, this document features methods for treating a mammal having cancer where the methods can include, or consist essentially of, administering an inhibitor of a mitochondrial polypeptide to a mammal identified as having a BRCA1-deficient cancer under conditions where the susceptibility of the cancer to treatment with a platinum compound or a PARP inhibitor increases. The mammal can be a human. The BRCA1-deficient cancer can be selected from the group consisting of ovarian cancers, breast cancers, pancreatic cancers, prostate cancers, skin cancers, renal cancers, liver cancers, stomach cancers, colon cancers, colorectal cancers, bladder cancers, and oral squamous cell cancers. The BRCA1-deficient cancer can be an ovarian cancer. The BRCA1-deficient cancer can be a breast cancer. The inhibitor of a mitochondrial polypeptide can be tigecycline. The method also can include administering the platinum compound to the mammal. The platinum compound can be cisplatin. The method also can include administering the PARP inhibitor to the mammal. The PARP inhibitor can be olaparib.

In another aspect, this document features methods for treating a mammal having cancer where the methods can include, or consist essentially of, (a) identifying the mammal as having a BRCA1-deficient cancer, and (b) administering an inhibitor of glucose transport to the mammal to increase the susceptibility of the cancer to treatment with a platinum compound or a PARP inhibitor. The mammal can be a human. The BRCA1-deficient cancer can be selected from the group consisting of ovarian cancers, breast cancers, pancreatic cancers, prostate cancers, skin cancers, renal cancers, liver cancers, stomach cancers, colon cancers, colorectal cancers, bladder cancers, and oral squamous cell cancers. The BRCA1-deficient cancer can be an ovarian cancer. The inhibitor of glucose transport can reduce or eliminate the expression and/or activity of a GLUT1 polypeptide. The inhibitor of glucose transport can be WZB117. The method also can include administering the platinum compound to the mammal. The platinum compound can be cisplatin. The method also can include administering the PARP inhibitor to the mammal. The PARP inhibitor can be olaparib.

In another aspect, this document features methods for treating a mammal having cancer where the methods can include, or consist essentially of, administering an inhibitor of glucose transport to a mammal identified as having a BRCA1-deficient cancer under conditions where the susceptibility of the cancer to treatment with a platinum compound or a PARP inhibitor increases. The mammal can be a human. The BRCA1-deficient cancer can be selected from the group consisting of ovarian cancers, breast cancers, pancreatic cancers, prostate cancers, skin cancers, renal cancers, liver cancers, stomach cancers, colon cancers, colorectal cancers, bladder cancers, and oral squamous cell cancers. The BRCA1-deficient cancer can be an ovarian cancer. The inhibitor of glucose transport can reduce or eliminate the expression and/or activity of a GLUT1 polypeptide. The inhibitor of glucose transport can be WZB117. The method also can include administering the platinum compound to the mammal. The platinum compound can be cisplatin. The method also can include administering the PARP inhibitor to the mammal. The PARP inhibitor can be olaparib.

In another aspect, this document features methods for treating a mammal having cancer where the methods can include, or consist essentially of, (a) identifying the mammal as having a NNMT overexpressing cancer, and (b) administering an OXPHOS inhibitor to the mammal to increase the susceptibility of the cancer to treatment with a platinum compound or a PARP inhibitor. The mammal can be a human. The NNMT overexpressing cancer can be selected from the group consisting of ovarian cancers, breast cancers, pancreatic cancers, prostate cancers, skin cancers, renal cancers, liver cancers, stomach cancers, colon cancers, colorectal cancers, bladder cancers, and oral squamous cell cancers. The NNMT overexpressing cancer can be an ovarian cancer. The OXPHOS inhibitor can be selected from the group consisting of VLX600 and ceritinib. The OXPHOS inhibitor can be VLX600. The method also can include administering the platinum compound to the mammal. The platinum compound can be cisplatin. The method also can include administering the PARP inhibitor to the mammal. The PARP inhibitor can be olaparib.

In another aspect, this document features methods for treating a mammal having cancer where the methods can include, or consist essentially of, administering an OXPHOS inhibitor to a mammal identified as having a NNMT overexpressing cancer under conditions where the susceptibility of the cancer to treatment with a platinum compound or a PARP inhibitor increases. The mammal can be a human. The NNMT overexpressing cancer can be selected from the group consisting of ovarian cancers, breast cancers, pancreatic cancers, prostate cancers, skin cancers, renal cancers, liver cancers, stomach cancers, colon cancers, colorectal cancers, bladder cancers, and oral squamous cell cancers. The NNMT overexpressing cancer can be an ovarian cancer. The OXPHOS inhibitor can be selected from the group consisting of VLX600 and ceritinib. The OXPHOS inhibitor can be VLX600. The method also can include administering the platinum compound to the mammal. The platinum compound can be cisplatin. The method also can include administering the PARP inhibitor to the mammal. The PARP inhibitor can be olaparib.

In another aspect, this document features methods for treating a mammal having cancer where the methods can include, or consist essentially of, (a) identifying the mammal as having a NNMT overexpressing cancer, and (b) administering an inhibitor of a mitochondrial polypeptide to the mammal to increase the susceptibility of the cancer to treatment with a platinum compound or a PARP inhibitor. The mammal can be a human. The NNMT overexpressing cancer can be selected from the group consisting of ovarian cancers, breast cancers, pancreatic cancers, prostate cancers, skin cancers, renal cancers, liver cancers, stomach cancers, colon cancers, colorectal cancers, bladder cancers, and oral squamous cell cancers. The NNMT overexpressing cancer can be an ovarian cancer. The NNMT overexpressing cancer can be a breast cancer. The inhibitor of a mitochondrial polypeptide can be tigecycline. The method also can include administering the platinum compound to the mammal. The platinum compound can be cisplatin. The method also can include administering the PARP inhibitor to the mammal. The PARP inhibitor can be olaparib.

In another aspect, this document features methods for treating a mammal having cancer where the methods can include, or consist essentially of, administering an inhibitor of a mitochondrial polypeptide to a mammal identified as having a NNMT overexpressing cancer under conditions where the susceptibility of the cancer to treatment with a platinum compound or a PARP inhibitor increases. The mammal can be a human. The NNMT overexpressing cancer can be selected from the group consisting of ovarian cancers, breast cancers, pancreatic cancers, prostate cancers, skin cancers, renal cancers, liver cancers, stomach cancers, colon cancers, colorectal cancers, bladder cancers, and oral squamous cell cancers. The NNMT overexpressing cancer can be an ovarian cancer. The NNMT overexpressing cancer can be a breast cancer. The inhibitor of a mitochondrial polypeptide can be tigecycline. The method also can include administering the platinum compound to the mammal. The platinum compound can be cisplatin. The method also can include administering the PARP inhibitor to the mammal. The PARP inhibitor can be olaparib.

In another aspect, this document features methods for treating a mammal having cancer where the methods can include, or consist essentially of, (a) identifying the mammal as having a NNMT overexpressing cancer, and (b) administering an inhibitor of glucose transport to the mammal to increase the susceptibility of the cancer to treatment with a platinum compound or a PARP inhibitor. The mammal can be a human. The NNMT overexpressing cancer can be selected from the group consisting of ovarian cancers, breast cancers, pancreatic cancers, prostate cancers, skin cancers, renal cancers, liver cancers, stomach cancers, colon cancers, colorectal cancers, bladder cancers, and oral squamous cell cancers. The NNMT overexpressing cancer can be an ovarian cancer. The inhibitor of glucose transport can reduce or eliminate the expression and/or activity of a GLUT1 polypeptide. The inhibitor of glucose transport can be WZB117. The method also can include administering the platinum compound to the mammal. The platinum compound can be cisplatin. The method also can include administering the PARP inhibitor to the mammal. The PARP inhibitor can be olaparib.

In another aspect, this document features methods for treating a mammal having cancer where the methods can include, or consist essentially of, administering an inhibitor of glucose transport to a mammal identified as having a NNMT overexpressing cancer under conditions where the susceptibility of the cancer to treatment with a platinum compound or a PARP inhibitor increases. The mammal can be a human. The NNMT overexpressing cancer can be selected from the group consisting of ovarian cancers, breast cancers, pancreatic cancers, prostate cancers, skin cancers, renal cancers, liver cancers, stomach cancers, colon cancers, colorectal cancers, bladder cancers, and oral squamous cell cancers. The NNMT overexpressing cancer can be an ovarian cancer. The inhibitor of glucose transport can reduce or eliminate the expression and/or activity of a GLUT1 polypeptide. The inhibitor of glucose transport can be WZB117. The method also can include administering the platinum compound to the mammal. The platinum compound can be cisplatin. The method also can include administering the PARP inhibitor to the mammal. The PARP inhibitor can be olaparib.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 shows a schematic of using VLX600 for treating a BRCA1-deficient cancer. The left panel shows VLX600 disrupting homologous recombination (HR) and sensitizing cells to platinum agents and PARP inhibitors. The right panel shows VLX600 selectively killing BRCA1-deficient or NNMT-overexpressing cancer cells as a monotherapy (left box) and VLX600 as a combination therapy having synergistic cytotoxicity (right box).

FIG. 2 contains graphs showing that VLX600 sensitizes ovarian cancer cells to cisplatin and the PARP inhibitor olaparib. OVCAR-8 (A and C) or PEA1 (B and D) cells were plated, allowed to adhere overnight, and treated with vehicle or indicated concentrations of VLX600 plus olaparib (A and B) or cisplatin (C and D) for 10 days, stained with Coomassie Blue, and the colonies were counted manually. A representative experiment from 3 independent experiments is shown.

FIG. 3 shows that VLX600 disrupts HR but not cell cycle or DNA replication. (A) OVCAR-8 cells that have stably integrated DR-GFP HR substrate were transfected with pCβASceI plasmid, allowed to adhere on culture plates for 6 hours, and treated with indicated concentrations of VLX600 for 72 hours. The cells were analyzed for GFP fluorescence by flow cytometry. (B) Same as in A, but cells were supplemented with or without FeCl₂ or FeCl₃. (C) Cell cycle profile of vehicle- or 100 nM VLX600-treated cells used in A. (D) Cell cycle profile of vehicle- or 100 nM VLX600-treated cells used in A. (D) OVCAR-8 cells were treated with vehicle or 100 nM VLX600 for 72 hours, and DNA replication was measured by EdU incorporation using Click-iT Plus EdU Alexa Fluor 594 Flow Cytometry Assay Kit (Thermofisher) and flow cytometry.

FIG. 4 shows that VLX600 is synthetically lethal with BRCA1 deficiency. OVCAR-8 (A and C) and PEA1 (B and D) cells were transfected with control luciferase (Luc), or two independent BRCA1 or BRCA2 siRNAs. 48 hours later, the cells were trypsinized, re-plated, and cultured for 24 hours. The indicated concentrations of VLX600 were then added, and the cells were cultured for 10 days, stained with Coomassie Blue, and the colonies were counted manually. A representative experiment from 3 independent experiments is shown.

FIG. 5 shows that BRCA1 depletion increases abundance of both nicotinamide N-methyltransferase (NNMT) mRNA and of NNMT polypeptide. Ovarian cancer cells, OVCAR-8 and PEO1, were transfected with control (Luc) and two different siRNAs targeting BRCA1, BRCA2, and RAD51, and NNMT mRNA and protein levels were examined by qRT-PCR (A) and Western blotting (B), respectively, 48 hours later.

FIG. 6 shows that BRCA1 depletion reprograms metabolism. OVCAR-8 cells were transfected with control luciferase, BRCA1, BRCA2, or RAD51 siRNAs. 48 hours later, (A) basal oxygen consumption rate was determined by Seahorse X-24 analyzer (Agilent), (B) rate of ATP production was determined by HPLC (Merck), and (C) levels of ROS were determined by cellular ROS detection assay kit (Abcam) and flow cytometry.

FIG. 7 shows that NNMT depletion reverses metabolic phenotype induced by BRCA1 depletion and that NNMT overexpression alone reduces oxygen consumption and ATP levels. (A and B) Rescue of ATP levels (D) and oxygen consumption rate in BRCA1 and NNMT co-depleted OVCAR-8 cells. (C and D) Exogenous overexpression of NNMT phenocopies the effect of BRCA1 depletion in oxygen consumption rate and ATP levels.

FIG. 8 shows that BRCA1 occupies the NNMT promoter. ChIP was performed on OVCAR-8 cells using anti-BRCA1 or IgG control antibody and primers specific for the NNMT promoter.

FIG. 9 shows that BRCA1 depletion sensitizes to VLX600 and that BRCA1-deficient cells are even more sensitive to VLX600 combined with olaparib or cisplatin. OVCAR-8 cells (A and B) or PEA1 cells (C) that were transfected with control luciferase (Luc) or BRCA1 siRNAs, and clonogenic assays with olaparib alone or olaparib plus 30 nM VLX600 (A and C) or cisplatin alone or cisplatin plus 30 nM VLX600 (B) were performed as described in FIG. 4. Insets show higher-magnification plots of BRCA1-depleted cells.

FIG. 10 shows that CDK12 depletion downregulates BRCA1, which causes decreased ATP levels and suppresses mitochondrial respiration. (A) Analysis of ATP levels in CDK12-depleted cells. OVCAR-8 (top panel) and PEA1 (bottom panel) cells were transfected with control luciferase (siLuc) or CDK12 siRNAs (siCDK12 #1 and siCDK12 #2). 48 hours after transfection, ATP levels were measured (right panels), and CDK12 and HPS90 levels were analyzed by immunoblotting (left panels). Relative ATP levels were normalized to cells transfected with siLuc. Data are means±SEM, n=3 independent experiments. *P<0.05, **P<0.01, unpaired t test. (B) Effect of BRCA1 depletion on oxygen consumption rate (OCR). OVCAR-8 or PEA1 cells were transfected with control siLuc or CDK12 siRNAs. 48 hours later, OCRs were measured under basal conditions and following the sequential additions of oligomycin, FCCP, and rotenone/antimycin A using a Seahorse XFp extracellular flux analyzer. Data are representative of 3 independent experiments. (C) BRCA1 depletion also disrupts OCR. OVCAR-8 and PEA1 cells were transfected with control siLuc or BRCA1 siRNAs (siBRCA1 #1 and siBRCA1 #2). 48 hours later, BRCA1 and HSP90 were analyzed by immunoblotting (left panels), and OCR was measured as described in B (right panels). Data are means±SEM, n=3 independent experiments. *P<0.05, **P<0.01, ***P<0.001, unpaired t test. (D) Ectopic BRCA1 expression rescues the OCR defect induced by BRCA1. OVCAR-8 (top panel) and PEA1 (bottom panel) cells were transfected with siLuc or CDK12 siRNAs plus empty vector (EV) or SFB-BRCA1 plasmid (SFB-BRCA1). OCR was measured as described in B. Data are means±SEM, n=3 independent experiments. *P<0.05, **P<0.01, ***P<0.001, unpaired t test.

FIG. 11 shows results of analyses of mitochondrial metabolism. (A) CDK12 depletion does not affect glycolysis. OVCAR-8 and PEA1 cells were transfected with control luciferase (siLuc) or CDK12 siRNAs. 48 hours later, extracellular acidification rate (ECAR), an indicator of glycolysis, was measured under basal conditions or following the addition of glucose (fuel for glycolysis), oligomycin (oligo, an ATP synthase blocker), and 2-deoxyglucose (2-DG, an inhibitor of glycolysis) using a Seahorse XFp extracellular flux analyzer. Data are representative of 3 independent experiments for each cell line. (B) BRCA1 depletion reduces ATP levels. OVCAR-8 and PEA1 cells were transfected with siLuc or BRCA1 siRNAs. 48 hours after transfection, ATP levels were measured and normalized to cells transfected with siLuc siRNA. Data are means±SEM, n=3 independent experiments. *P<0.05, **P<0.01, ***P<0.001, unpaired t test. (C and D) Glycolysis was unchanged by BRCA1 depletion. OVCAR-8 and PEA1 cells were transfected with siLuc or BRCA1 siRNAs. 48 hours later, ECAR was measured as described above. Data are representative of 3 independent experiments for each cell line. (E and F) OVCAR-8 cells were transfected with control luciferase (siLuc) plus empty vector (EV) or SFB-BRCA1 plasmids or CDK12 siRNA plus EV or SFB-BRCA1 plasmids. 48 hours later, the cells were analyzed by qPCR for BRCA1 and CDK12 mRNA levels, which are expressed relative to GAPDH mRNA levels as an internal control.

FIG. 12 shows that depletion of other proteins (BRCA2 and RAD51) involved in homologous recombination (HR) do not disrupt oxygen consumption rate (OCR), thus demonstrating that BRCA1's effects on metabolism are not the result of an HR defect. (A and C) BRCA2 or RAD51 depletion did not alter mitochondrial respiration. OVCAR-8 cells were transfected with siLuc, BRCA2 (A), or RAD51 (C) siRNAs, and OCR was measured as described above (left panels). To check the efficiency of the siRNA knockdown, the cells were immunoblotted for BRCA2, RAD51, and HSP90 (right panels). (B and D) BRCA2 or RAD51 depletion sensitizes cells to PARPi. A portion of the cells used in A and C were subjected to colony formation assays using olaparib, a PARP inhibitor (as a positive control for HR deficiency). Data are representative of 3 independent experiments.

FIG. 13 shows that BRCA1 depletion induces NNMT upregulation, which is responsible for the decreased OCR. (A and B) OVCAR-8, PEA1, and OVCAR-5 cells were transfected with control luciferase (siLuc), BRCA1, or NNMT siRNAs. 48 hours later, the cells were analyzed by qPCR for NNMT and BRCA1 mRNA levels, which are expressed relative to GAPDH mRNA levels as an internal control (A), and immunoblotted for NNMT, BRCA1, and HSP90 (B). Data are means±SEM, n=3 independent experiments. **P<0.01, ***P<0.001, unpaired t test. (C) Co-depletion of NNMT reverses the OCR defect induced by BRCA1 depletion. OVCAR-8 (top panel) and PEA1 (bottom panel) cells were transfected with siLuc, BRCA1, or NNMT siRNAs individually or co-transfected with BRCA1 and NNMT siRNAs. OCR was measured as described in FIG. 10B. Data are means±SEM, n=3 independent experiments. *P<0.05, **P<0.01, ***P<0.001, unpaired t test. (D) NNMT overexpression phenocopies the OCR defect induced by BRCA1 depletion. OVCAR-8 cells were transiently transfected with empty vector (EV) or a plasmid that expresses Myc-DDK-tagged NNMT. After 24 hours, OCR was analyzed as described in FIG. 10B and cells were immunoblotted for NNMT and HSP90. Data are means±SEM, n=3 independent experiments. *P<0.05, **P<0.01, ***P<0.001, unpaired t test.

FIG. 14 shows that BRCA1 depletion sensitizes ovarian cancer cells to agents that disrupt metabolism. (A-C) OVCAR-8 and PEA1 cells were transfected with control luciferase (siLuc) or BRCA1 siRNAs. 48 hours later, the cells were trypsinized, immunoblotted for BRCA1 and HSP90 (bottom panels), and subjected to colony formation assays. The indicated concentrations of VLX600 (A), tigecycline (B), or WZB117 (C) were added 12 hours after plating, and the cells were cultured for 8-10 days to allow colony formation. Data are representative of 3 independent experiments. Error bars: means±SEM.

FIG. 15 shows that depletion of other proteins (BRCA2 and RAD51) involved in homologous recombination (HR) does not sensitize ovarian cancer cells to VLX600. (A and B) OVCAR-8 cells were transfected with control luciferase (siLuc), BRCA2 (A) or RAD51 (B) siRNAs and colony formation assays were performed with VLX600 (left panels) or the PARP inhibitor olaparib, which served as a positive control to show that HR was disabled (right panels).

FIG. 16 shows that NNMT overexpression sensitizes ovarian cancer cells to agents that disrupt metabolism. (A-D) Clones of OVCAR-8 cells stably transfected with empty vector (pcDNA3) or the Myc-DDK-NNMT expression plasmid were subjected to immunoblotting for NNMT and HSP90 (A), examined for OCR as described in FIG. 10B (B), and treated with VLX600 (C) or tigecycline (D), which were added 12 hours after plating the cells. The cells were cultured for 8-10 days to allow colony formation. Data are representative of 3 independent experiments. Error bars: means±SEM.

FIG. 17 shows that CDK12 depletion downregulates BRCA1, which decreased ATP levels and suppressed mitochondrial respiration. (A) Another presentation of the results in FIG. 10A using tubulin blot is as a loading control in place of HSP90. Absolute ATP concentrations (right panels) replace ATP levels expressed as % siLuc. (B) Another presentation of the results in FIG. 10C (left panels) using tubulin blot is as a loading control in place of HSP90.

FIG. 18 shows that CDK12 and BRCA1 depletion reduces ATP and increases ADP levels. (A and B) OVCAR-8 cells were transfected with control luciferase siRNA (siLuc), two different non-targeting siRNAs (siNT #3 and siNT #5), CDK12 siRNAs (siCDK12 #1 and siCDK12 #2) or BRCA1 siRNAs (siBRCA1 #1 and siBRCA1 #2). 48 hours after transfection, ATP (A) and ADP (B) levels were measured. Data are means±SEM, n=3 independent experiments. *P<0.05, **P<0.01, unpaired t test, comparing to siLuc.

FIG. 19 shows that CDK12 and BRCA1 depletion reduce ATP levels in short-term ex vivo-cultured HGSOC patient-derived xenograft (PDX) tumors (A and B), that BRCA1 re-expression in BRCA1 deficient cells increases OCR (C), and that CDK12 and BRCA1 depletion does not affect mitochondrial DNA levels (D). (A and B) HGSOC PDX tumors freshly excised from mice were disaggregated into single-cell suspensions, electroporated with control siRNAs (Luc, NT #1), CDK12, or BRCA1 siRNAs. 48 hours later, cells were analyzed for BRCA1 and CDK12 mRNA levels by qRT-PCR (B) and ATP content (A), and ATP levels were normalized to Luc siRNA-transfected cells. Data are means±SEM, n=2 independent PDX tumors. *P<0.05, **P<0.01 unpaired t test, comparing to siLuc. (C) Re-expression of BRCA1 increases OCR in COV362 cells. OCR of COV362 cells stably overexpressing SFB-BRCA1 or empty vector (pCDNA3.1) was measured. Data are representative of 3 independent experiments. Error bars indicate Standard Error of duplicate wells from a single experiment. Representative immunoblots of exogenous SFB-BRCA1 in COV362-EV and COV362-SFB-BRCA1 stable cells using FLAG and tubulin antibodies are shown. (D) Depletion of CDK12 and BRCA1 do not reduce mitochondrial DNA content. Mean mitochondrial DNA copy number in OVCAR-8 cells transfected with control luciferase (Luc), two different non-targeting siRNAs (NT #3 and NT #5), CDK12 siRNAs (siCDK12 #1 and siCDK12 #2), or BRCA1 siRNAs (siBRCA1 #1 and siBRCA1 #2) was determined 48 hours after transfection using the human mitochondrial DNA monitoring primer set and qRT-PCR.

FIG. 20 shows that BRCA1 and CDK12 mRNA and protein levels are inversely correlated with NNMT mRNA and protein. (A) Another presentation of the data shown in FIG. 13B using tubulin blot as a loading control in place of HSP90. (B) Scatter plots of NNMT mRNA expression as a function of BRCA1 and CDK12 mRNA expression in HGSOC tumors from patients and PDX models (left two panels). Scatter plots of NNMT protein as a function of BRCA1 mRNA and CDK12 protein levels in HGSOC tumors from patients. Spearman or Pearson correlations are shown in the images. (C) Lower panel in (C) is another presentation of the data shown in FIG. 13D with an updated immunoblot for NNMT.

FIG. 21 shows a BRCA1 gene map with known mRNA transcripts (A), the effect of BRCA1 siRNAs #1 and #2 on different exon expression (B and C), and association of BRCA1 with the NNMT promoter (D and E). (A) The target sites of BRCA1 siRNAs (siBRCA1 #1 and siBRCA1 #2) and the location of qRT-PCR primer sets on BRCA1 (ENSG00000012048.15; gtexportal.org/home/gene/BRCA1#ptvBlock) exons. BRCA1 Primer #1 and BRCA1 Primer #2 are not shown because they are specific for the BRCA1 Δ11q transcript. (B) BRCA1 mRNA levels, which are expressed relative to GAPDH mRNA levels, in BRCA1 siRNA-transfected OVCAR-8 cells analyzed by qRT-PCR using primers indicated in (A). Data are means±SEM, n=3 independent experiments. ***P<0.001, unpaired t test. (C) BRCA1 occupies the NNMT promoter. ChIP assays in OVCAR-8 and PEA1 cells using anti-BRCA1 or IgG control antibodies and primers specific for the NNMT promoter. (D) ChIP assay as in FIG. 8, but using mouse monoclonal anti-FLAG antibody (F1804, Sigma) in lysates from empty vector or SFB-BRCA1-transfected OVCAR-8 cells. Immunoblot of SFB-BRCA1 using rabbit monoclonal DYKDDK (SEQ ID NO:18) antibody (recognizes same sequence as FLAG®, 1:1000, #14793, Cell Signaling Technology) is shown. Tubulin was used as loading control. Data are means±SEM, n=3 independent experiments. *P<0.05, **P<0.01, unpaired t test.

FIG. 22 shows that BRCA1 depletion sensitizes ovarian cancer cells to agents that disrupt metabolism. OVCAR-8 (A) and PEA1 (B) cells were transfected with control luciferase (Luc) or BRCA1 siRNAs. 48 hours later, the cells were trypsinized, plated in 6-well plates, and subjected to colony formation assays. The indicated concentrations of ceritinib was added 12 hours after plating. Data are representative of 3 independent experiments. Error bars: means±SEM.

FIG. 23 shows that NNMT overexpression reduces OCR. (A) Another presentation of results shown in FIGS. 16A and B using tubulin blot is as a loading control in place of HSP90. (B) Bar graph is another presentation of OCR data shown in FIG. 16B. Data in (B) are means±SEM, n=3 independent experiments, **P<0.01, unpaired t test.

FIG. 24 shows that NNMT overexpression sensitizes ovarian cancer cells to agents that disrupt metabolism. Clones of OVCAR-8 cells stably transfected with empty vector (pcDNA3) or the Myc-DDK-NNMT expression plasmid (shown in FIG. 23) were plated as single cells, allowed to adhere for 12 hours, and treated with ceritinib. The cells were cultured for 8-10 days in the presence of ceritinib to allow colony formation. Data are means±SEM from triplicate wells, n=1 experiment.

FIG. 25 shows that NNMT overexpression sensitizes ovarian cancer cells to agents that disrupt metabolism. Clones of OV90 cells stably transfected with empty vector (pcDNA3) or the Myc-DDK-NNMT expression plasmid were plated as single cells, allowed to adhere for 12 hours, and then treated with VLX600. The cells were cultured for 8-10 days in the presence of VLX600 to allow colony formation. Data are means±SEM from triplicate wells, n=1 experiment.

FIG. 26 shows that NNMT overexpression sensitizes colon cancer cells to agents that disrupt metabolism. Clones of HCT-116 colon cancer cells stably transfected with empty vector (pcDNA3) or the Myc-DDK-NNMT expression plasmid (Myc-NNMT Clone #4, #8, #22) were plated as single cells, allowed to adhere for 12 hours, and then treated with ceritinib. The cells were cultured for 8-10 days in the presence of VLX600 to allow colony formation. Data are means±SEM from triplicate wells, n=1 experiment.

FIG. 27 shows that VLX600 further reduces ATP levels in NNMT overexpressing and BRCA1-depleted cells causing cell death. (A) OVCAR-8 cells transfected with empty vector (EV) or stably expressing Myc-DDK-NNMT were treated with 50 nM VLX600 for 24 hours, and OCR was measured. (B) Control luciferase (siLuc)- or BRCA1 siRNA-transfected OVCAR-8 cells were treated with indicated concentrations of VLX600 for 24 hours and OCR was measured. (C) A schematic for how NNMT overexpression creates a metabolic liability by reducing ATP levels, which are further reduced by OXPHOS inhibitors. Data in (A and B) are means±SEM, n=3 independent experiments, *P<0.05, **P<0.01, unpaired t test.

FIG. 28 shows that CDK12 and BRCA1 depletion does not further suppress ATP levels in NNMT-overexpressing cells. (A) Stably transfected OVCAR-8-EV and OVCAR-8-Myc-DDK-NNMT (from FIG. 23) were transfected with control luciferase (Luc), non-targeting siRNA #3, BRCA1, or CDK12 siRNAs. 48 hours later, ATP levels were measured and cells were immunoblotted for BRCA1, CDK12, NNMT, and tubulin. Data are means±SEM, n=3 independent experiments. *P<0.05, unpaired t test. (B-C) NNMT overexpression does not affect cell proliferation. OVCAR-8-EV and OVCAR-8-Myc-DDK-NNMT cells (B) and OVCAR-8 cells transfected with control luciferase (Luc), non-targeting siRNAs #3 and #5, and BRCA1 siRNAs (C) were analyzed using the CyQUANT® Cell Proliferation Assay Kit, 1, 2, and 3 days after plating. Data are means±SEM, n=3 independent experiments. *P<0.05, **P<0.01, unpaired t test.

### DETAILED DESCRIPTION

This document provides methods and materials involved in treating mammals in need thereof (e.g., mammals having a cancer such as a BRCA1-deficient cancer, a CDK12-deficient cancer, and/or a NNMT overexpressing cancer). For example, a mammal having cancer (e.g., a BRCA1-deficient cancer, a CDK12-deficient cancer, and/or a NNMT overexpressing cancer) can be treated by administering one or more OXPHOS inhibitors to the mammal. In some cases, one or more OXPHOS inhibitors can be used in combination with one or more PARP inhibitors, one or more platinum compounds, and/or one or more chemotherapy agents that induce DNA crosslinks to treat cancer (e.g., a BRCA1-deficient cancer, a CDK12-deficient cancer, and/or a NNMT overexpressing cancer).

Any type of mammal having a cancer can be treated as described herein. Examples of mammals that can be treated by administering one or more OXPHOS inhibitors (e.g., VLX600), and, optionally, one or more PARP inhibitors, one or more platinum compounds, and/or one or more chemotherapy agents that induce DNA crosslinks, include, without limitation, humans, non-human primates (e.g., monkeys), dogs, cats, horses, cows, pigs, sheep, mice, and rat. In some cases, a human can be treated by administering one or more OXPHOS inhibitors (e.g., VLX600), and, optionally, one or more PARP inhibitors, one or more platinum compounds, and/or one or more chemotherapy agents that induce DNA crosslinks.

When treating a mammal having a cancer as described herein, the cancer can be any type of cancer. In some cases, a cancer treated as described herein can be a BRCA1-deficient cancer, a CDK12-deficient cancer, and/or a cancer that overexpresses a NNMT polypeptide. A cancer treated as described herein can be a primary cancer or a metastatic cancer. A cancer treated as described herein can be a hormone receptor positive cancer or a hormone receptor negative cancer. In some cases, a cancer treated as described herein can include one or more solid tumors. In some cases, a cancer treated as described herein can be a cancer in remission. In some cases, a cancer treated as described herein can include quiescent (e.g., dormant or non-dividing) cancer cells. In some cases, a cancer treated as described herein can be cancer that has escaped chemotherapy and/or has been non-responsive to chemotherapy. In some cases, a cancer treated as described herein can be a hypoxic cancer. In some cases, a cancer treated as described herein can be a KRAS-dependent cancer. In some cases, a cancer treated as described herein can be a homologous recombination DNA repair-proficient cancer. In some cases, a cancer treated as described herein can be a cancer that overexpresses a NNMT polypeptide. Examples of cancers that can be treated by administering one or more OXPHOS inhibitors (e.g., VLX600), and, optionally, one or more PARP inhibitors, one or more platinum compounds, and/or one or more chemotherapy agents that induce DNA crosslinks, include, without limitation, ovarian cancers, breast cancers, pancreatic cancers, prostate cancers, skin cancers, renal cancers, liver cancers, stomach cancers, colon cancers, colorectal cancers, bladder cancers, and oral squamous cell cancers. In some cases, a mammal (e.g., a human) having ovarian cancer can be treated by administering one or more OXPHOS inhibitors (e.g., VLX600), and, optionally, one or more PARP inhibitors, one or more platinum compounds, and/or one or more chemotherapy agents that induce DNA crosslinks.

In some cases, a mammal can be identified as having a cancer (e.g., a BRCA1-deficient cancer, a CDK12-deficient cancer, and/or a NNMT overexpressing cancer). Any appropriate method can be used to identify a mammal as having a cancer. For example, imaging techniques and/or biopsy techniques can be used to identify mammals (e.g., humans) having cancer.

When a mammal has a BRCA1-deficient cancer, the BRCA1-deficient cancer can be any appropriate BRCA1-deficient cancer. In some cases, a BRCA1-deficient cancer can refer to any cancer that includes one or more cancer cells having one or more modifications in a BRCA1 nucleic acid (e.g., a nucleic acid encoding a BRCA1 polypeptide) and/or one or more modifications in a BRCA1 polypeptide that alter cancer cell metabolism. A modification can alter any appropriate type of cancer cell metabolism. In some cases, a modification does not affect glycolysis. Examples of metabolic alterations that can be seen in cells in a BRCA1-deficient cancer include, without limitation, reduced levels of adenosine-5′-triphosphate (ATP), increased levels of reactive oxygen species (ROS), and reduced OXPHOS. In some cases, a BRCA1-deficient cancer can include one or more cancer cells having one or more modifications in a BRCA1 nucleic acid and/or one or more modifications in a BRCA1 polypeptide that can reduce OXPHOS. A modification can be any appropriate modification. A modification in a BRCA1 nucleic acid or a modification in a BRCA1 polypeptide refers to any change in a BRCA1 nucleic acid sequence or a change in a BRCA1 polypeptide sequence relative to a normal (e.g., wild type) BRCA1 sequence. Any appropriate method can be used to identify the presence or absence of a modification in a BRCA1 nucleic acid and/or a BRCA1 polypeptide. In some cases, one or more sequencing techniques (e.g., nucleic acid sequencing techniques or polypeptide sequencing techniques) can be used to identify the presence or absence of a modification in a BRCA1 nucleic acid and/or a BRCA1 polypeptide. Examples of modifications in a BRCA1 nucleic acid and/or a BRCA1 polypeptide that can alter cancer cell metabolism include, without limitation, epigenetic silencing of BRCA1 (e.g., due to promoter methylation), genomic deletions that include all or part of a BRCA1 nucleic acid, modifications that introduce premature stop codons (e.g., frameshift and nonsense mutations), modifications that alter the coding sequence (e.g., missense mutations), and modifications that lead to truncated BRCA1 polypeptides.

In some cases, a BRCA1-deficient cancer can include one or more cancer cells having one or more modifications as described elsewhere (see, e.g., the cBioPortal for Cancer Genomics; Gao et al., Sci Signal. 2013; 6(269):11; Cerami et al., Cancer Discov. 2012; 2(5); 401-4; and Elstrodt et al., 2006 Cancer Res 66:41-45).

In some cases, a BRCA1-deficient cancer can refer to any cancer that includes reduced or eliminated BRCA1 polypeptide expression and/or reduced or eliminated BRCA1 polypeptide activity. A reduced level of BRCA1 polypeptide expression or BRCA1 polypeptide activity refers to any level of BRCA1 polypeptide expression or BRCA1 polypeptide activity that is lower than the median level of BRCA1 polypeptide expression or BRCA1 polypeptide activity typically observed in a sample (e.g., a control sample) from one or more healthy mammals (e.g., healthy humans) and/or from one or more healthy tissues (e.g., healthy human tissues). Control samples can include, without limitation, samples from mammals that do not have cancer, cell lines originating from mammals that do not have cancer, non-tumorigenic cell lines, and adjacent normal tissue. It will be appreciated that comparable samples are used when determining whether or not a particular level is a reduced level. An eliminated level of BRCA1 polypeptide expression or BRCA1 polypeptide activity refers to any non-detectable level of BRCA1 polypeptide expression or BRCA1 polypeptide activity. Any appropriate method can be used to determine whether or not a cancer has reduced or eliminated BRCA1 polypeptide expression and/or BRCA1 polypeptide activity. For example, the presence, absence, level, or activity of BRCA1 polypeptides can be detected in a sample (e.g., a tumor sample such as a cancer biopsy) obtained from a mammal to determine if the mammal has a BRCA1-deficient cancer. For example, western blotting, reverse-transcription polymerase chain reaction (RT-PCR), spectrometry methods (e.g., high-performance liquid chromatography (HPLC) and liquid chromatography-mass spectrometry (LC/MS)), enzyme-linked immunosorbent assay (ELISA), and the ability of BRCA1 polypeptides to bind with nucleic acid (e.g., deoxyribonucleic acid (DNA)) can be used to determine whether or not a sample contains a reduced or eliminated levels BRCA1 polypeptide expression or BRCA1 polypeptide activity. In some cases, when reduced or eliminated BRCA1 polypeptide expression and/or reduced or eliminated BRCA1 polypeptide activity is/are detected in a sample obtained from a mammal having cancer, the mammal can be identified as having a BRCA1-deficient cancer.

When a mammal has a CDK12-deficient cancer, the CDK12-deficient cancer can be any appropriate CDK12-deficient cancer. In some cases, a CDK12-deficient cancer can refer to any cancer that includes one or more cancer cells having one or more modifications in a CDK12 nucleic acid (e.g., a nucleic acid encoding a CDK12 polypeptide) and/or one or more modifications in a CDK12 polypeptide that alter cancer cell metabolism. A modification can alter any appropriate type of cancer cell metabolism. In some cases, a modification does not affect glycolysis. Examples of metabolic alterations that can be seen in cells in a CDK12-deficient cancer include, without limitation, reduced levels of ATP, increased levels of ROS, and reduced OXPHOS. In some cases, a CDK12-deficient cancer can include one or more cancer cells having one or more modifications in a CDK12 nucleic acid and/or one or more modifications in a CDK12 polypeptide that can reduce OXPHOS. A modification can be any appropriate modification. A modification in a CDK12 nucleic acid or a modification in a CDK12 polypeptide refers to any change in a CDK12 nucleic acid sequence or a change in a CDK12 polypeptide sequence relative to a normal (e.g., wild type) CDK12 sequence. Any appropriate method can be used to identify the presence or absence of a modification in a CDK12 nucleic acid and/or a CDK12 polypeptide. In some cases, one or more sequencing techniques (e.g., nucleic acid sequencing techniques or polypeptide sequencing techniques) can be used to identify the presence or absence of a modification in a CDK12 nucleic acid and/or a CDK12 polypeptide. Examples of modifications in a CDK12 nucleic acid and/or a CDK12 polypeptide that can alter cancer cell metabolism include, without limitation, epigenetic silencing of CDK12 (e.g., due to promoter methylation), genomic deletions that include all or part of a CDK12 nucleic acid, modifications that introduce premature stop codons (e.g., frameshift and nonsense mutations), modifications that alter the coding sequence (e.g., missense mutations), and modifications that lead to truncated CDK12 polypeptides.

In some cases, a CDK12-deficient cancer can refer to any cancer that includes reduced or eliminated CDK12 polypeptide expression and/or reduced or eliminated CDK12 polypeptide activity. A reduced level of CDK12 polypeptide expression or CDK12 polypeptide activity refers to any level of CDK12 polypeptide expression or CDK12 polypeptide activity that is lower than the median level of CDK12 polypeptide expression or CDK12 polypeptide activity typically observed in a sample (e.g., a control sample) from one or more healthy mammals (e.g., healthy humans) and/or from one or more healthy tissues (e.g., healthy human tissues). Control samples can include, without limitation, samples from mammals that do not have cancer, cell lines originating from mammals that do not have cancer, non-tumorigenic cell lines, and adjacent normal tissue. It will be appreciated that comparable samples are used when determining whether or not a particular level is a reduced level. An eliminated level of CDK12 polypeptide expression or CDK12 polypeptide activity refers to any non-detectable level of CDK12 polypeptide expression or CDK12 polypeptide activity. Any appropriate method can be used to determine whether or not a cancer has reduced or eliminated CDK12 polypeptide expression and/or CDK12 polypeptide activity. For example, the presence, absence, level, or activity of CDK12 polypeptides can be detected in a sample (e.g., a tumor sample such as a cancer biopsy) obtained from a mammal to determine if the mammal has a CDK12-deficient cancer. For example, western blotting, RT-PCR, and spectrometry methods (e.g., HPLC and LC/MS), ELISA can be used to determine whether or not a sample contains a reduced or eliminated levels CDK12 polypeptide expression or CDK12 polypeptide activity. In some cases, when reduced or eliminated CDK12 polypeptide expression and/or reduced or eliminated CDK12 polypeptide activity is/are detected in a sample obtained from a mammal having cancer, the mammal can be identified as having a CDK12-deficient cancer.

When a mammal has a NNMT overexpressing cancer, the NNMT overexpressing cancer can be any appropriate NNMT overexpressing cancer. For example, a NNMT overexpressing cancer that can be treated as described herein can be a cancer that includes one or more cancer cells that express an elevated level of a NNMT polypeptide as compared to control cells such as non-cancer cells of the same type. In some cases, a NNMT overexpressing cancer that can be treated as described herein can be a cancer that includes one or more cancer cells that express at least 5 (e.g., at least 5, 10, 25, 35, 45, 50, 55, 65, 75, 90, or more) percent more of NNMT polypeptide than control cells such as non-cancer cells of the same type. Examples of NNMT polypeptides (and nucleic acids encoding such polypeptides) include, without limitation, those set forth in the National Center for Biotechnology Information (NCBI) databases at, for example, accession nos. NM_006169 and NP_006160.

In some cases, a mammal having a cancer (e.g., a BRCA1-deficient cancer, a CDK12-deficient cancer, and/or a NNMT overexpressing cancer) can be administered, or instructed to self-administer, one or more agents that can inhibit metabolism (e.g., mitochondrial metabolism). Examples of agents that can inhibit mitochondrial metabolism and be used as described herein include, without limitation, VLX600, ceritinib, brigatinib, and tigecycline. An agent that can inhibit metabolism can inhibit any appropriate metabolic pathway. In some cases, an agent that can inhibit one or more metabolic pathways can reduce or eliminate oxidative phosphorylation (e.g., can be an OXPHOS inhibitor). In some cases, an agent that can inhibit mitochondrial metabolism can reduce or eliminate translation (e.g., expression) and/or activity of a mitochondrial polypeptide (e.g., can be an inhibitor of a mitochondrial polypeptide such as Cox-1 and Cox-2; see, e.g., Škrtić et al., Cancer Cell, 20(5):674-688 (2011)). In some cases, an agent that can inhibit mitochondrial metabolism also can have one or more additional biological activities (e.g., antibiotic activity). For example, a mammal having a BRCA1-deficient cancer, a CDK12-deficient cancer, and/or a NNMT overexpressing cancer can be administered, or instructed to self-administer, VLX600. For example, a mammal having a BRCA1-deficient cancer, a CDK12-deficient cancer, and/or a NNMT overexpressing cancer can be administered, or instructed to self-administer, tigecycline or other OXPHOS inhibitors.

In some cases, a mammal having a cancer (e.g., a BRCA1-deficient cancer, a CDK12-deficient cancer, and/or a NNMT overexpressing cancer) can be administered, or instructed to self-administer, one or more agents that can inhibit glucose transport and/or one or more agents that inhibit glycolysis. An example of an agent that can inhibit glucose transport and be used as described herein to treat a mammal having a cancer (e.g., a BRCA1-deficient cancer, a CDK12-deficient cancer, and/or a NNMT overexpressing cancer) includes, without limitation, WZB117. In some cases, an agent that can inhibit glucose transport can reduce or eliminate translation (e.g., expression) and/or activity of a glucose transporter polypeptide (e.g., GLUT1). For example, a mammal having a BRCA1-deficient cancer, a CDK12-deficient cancer, and/or a NNMT overexpressing cancer can be administered, or instructed to self-administer, WZB117. An example of an agent that can inhibit aerobic glycolysis and be used as described herein to treat a mammal having a cancer (e.g., a BRCA1-deficient cancer, a CDK12-deficient cancer, and/or a NNMT overexpressing cancer) includes, without limitation, lonidamine. In some cases, an agent that can inhibit aerobic glycolysis can reduce or eliminate ATP levels.

In some cases, a mammal having a BRCA1-deficient cancer and/or a CDK12-deficient cancer also can be administered, or instructed to self-administer, one or more NNMT polypeptides (or nucleic acid encoding a NNMT polypeptide). Exemplary NNMT polypeptide sequences (and the nucleic acids encoding such polypeptides) that can be used as described herein to treat a mammal having a cancer (e.g., a BRCA1-deficient cancer) can be as set forth in the National Center for Biotechnology Information (NCBI) databases at, for example, accession nos. NM_006169 and NP_006160. Nucleic acid encoding a NNMT polypeptide can be any appropriate nucleic acid. Nucleic acid can be DNA (e.g., a DNA construct), RNA (e.g., mRNA), or a combination thereof. In some cases, nucleic acid encoding a NNMT polypeptide can be incorporated into a vector (e.g., an expression vector or a viral vector) for delivery to cells within a mammal to be treated.

In some cases, a mammal having cancer (e.g., a BRCA1-deficient cancer, a CDK12-deficient cancer, and/or a NNMT overexpressing cancer) also can be administered, or instructed to self-administer, one or more PARP inhibitors, one or more platinum compounds, and/or one or more chemotherapy agents that induce DNA crosslinks. For example, a cancer treatment that includes administering VLX600 also can include administering one or more PARP inhibitors. Examples of PARP inhibitors that can be used as described herein include, without limitation, olaparib, rucaparib, veliparib, talazoparib, and niraparib. In some cases, a mammal having cancer (e.g., a BRCA1-deficient cancer, a CDK12-deficient cancer, and/or a NNMT overexpressing cancer) can be administered, or instructed to self-administer, VLX600 and olaparib. For example, a cancer treatment that includes administering VLX600 also can include administering one or more platinum compounds. Examples of platinum compounds include, without limitation, cisplatin, carboplatin, and oxaliplatin. In some cases, a mammal having cancer (e.g., a BRCA1-deficient cancer, a CDK12-deficient cancer, and/or a NNMT overexpressing cancer) can be administered, or instructed to self-administer, VLX600, cisplatin, and/or one or more chemotherapy agents that induce DNA crosslinks.

In some cases, a mammal having cancer (e.g., a BRCA1-deficient cancer, a CDK12-deficient cancer, and/or a NNMT overexpressing cancer) also can be administered, or instructed to self-administer, one or more additional cancer treatments. In some cases, a cancer treatment such as surgery can be used to treat a cancer (e.g., a BRCA1-deficient cancer, a CDK12-deficient cancer, and/or a NNMT overexpressing cancer). In some cases, a cancer treatment can include radiation treatment. In cases where two or more cancer treatments are administered to treat cancer as described herein, the two or more cancer treatments can be administered at the same time or independently.

In some cases, when treating a mammal having a cancer (e.g., a BRCA1-deficient cancer, a CDK12-deficient cancer, and/or a NNMT overexpressing cancer) as described herein, the treatment can reduce one or more symptoms of the cancer in the mammal. For example, the treatment can reduce the number of cancer cells within a mammal. For example, the treatment can reduce the size (e.g., volume) of one or more tumors within a mammal. In some cases, the size (e.g., volume) of one or more tumors present within a mammal does not increase.

In some cases, when treating a mammal having a cancer (e.g., a BRCA1-deficient cancer, a CDK12-deficient cancer, and/or a NNMT overexpressing cancer) as described herein, the treatment can increase survival of the mammal. For example, the treatment can increase progression-free survival of the mammal. For example, the treatment can increase overall survival of the mammal.

The invention will be further described in the following examples, which do not limit the scope of the invention described in the claims.

### EXAMPLES

### Example 1: VLX600 Disrupts HR and Synergizes with PARPi and Cisplatin in Ovarian Cancer Cells

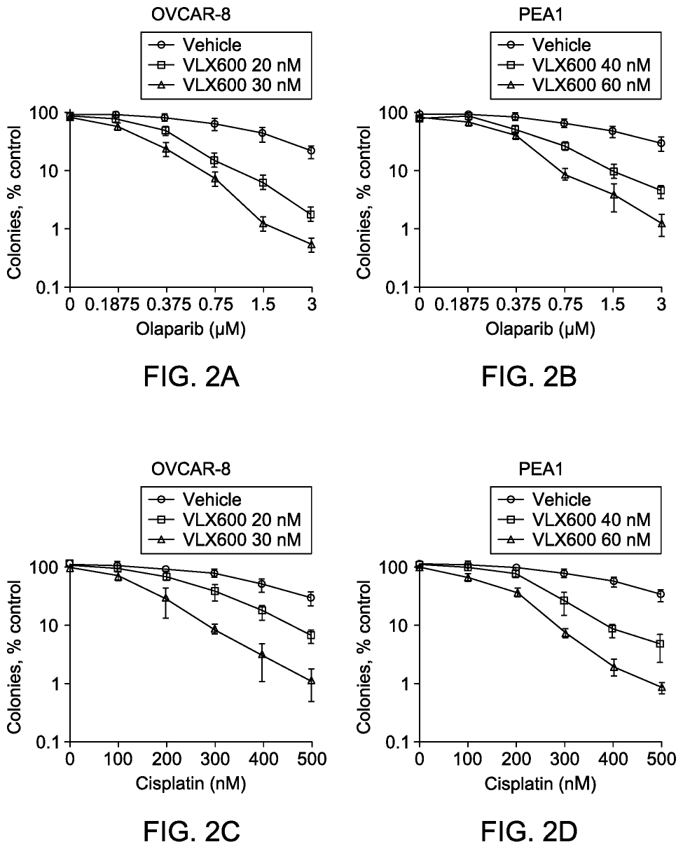

It was examined whether this agent synergized with cisplatin in ovarian cancer cell lines, first focusing on HR-proficient OVCAR-8 and PEA1 cells. It was observed that VLX600 sensitized to PARPi and cisplatin in multiple ovarian cancer cell lines at VLX600 concentrations that did not exhibit cytotoxicity as a single agent (FIG. 2).

### Example 2: VLX600 Disrupts HR

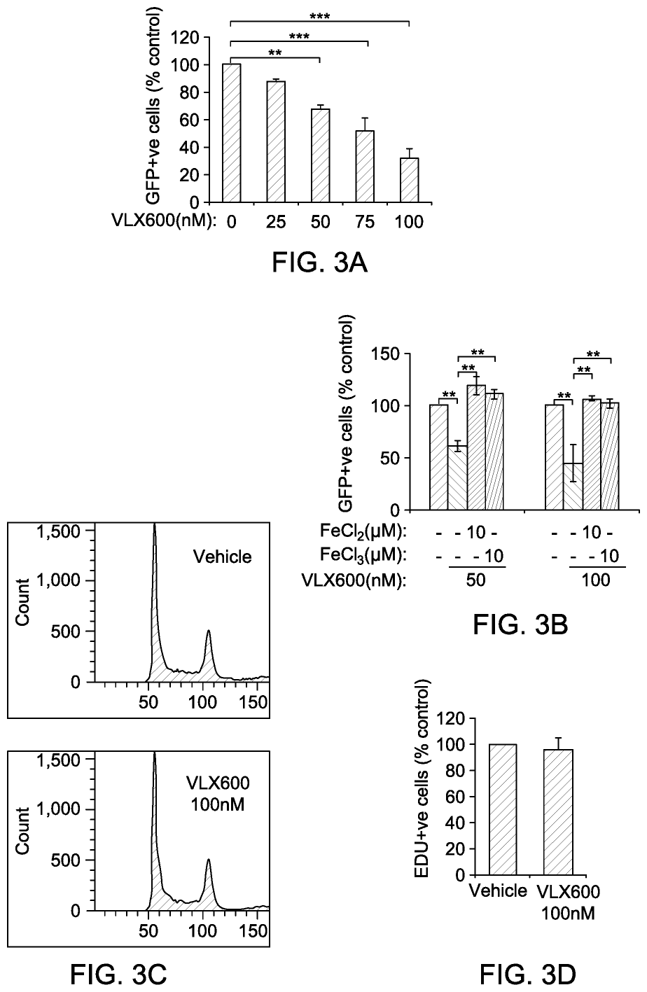

It was evaluated whether VLX600 affected HR using DR-GFP OVCAR-8 cells. As shown in FIG. 3A, VLX600 reduced HR at concentrations (e.g., 50-100 nM) that had no effect on survival (see FIG. 3), and the effect was reversible by addition of iron (FIG. 3B), indicating that it was due to the iron chelating activity of VLX600. The concentrations of VLX600 that sensitized to PARPi and that inhibited HR did not disrupt the cell cycle or DNA replication (FIG. 3C,D), indicating that these effects were not due to RNR inhibition at this very low concentration of VLX600 (note that RNR inhibition was observed at 10 Finally, the VLX600 concentrations were 3 orders of magnitude lower than the concentrations (approx. 100 μM) detected in the plasma of mice treated with VLX600, suggesting that concentrations of VLX600 that disrupt HR may be achievable. However, the mechanism by which VLX600 disrupts HR is unknown.

### Example 3: VLX600 is Synthetically Lethal with BRCA1 Deficiency

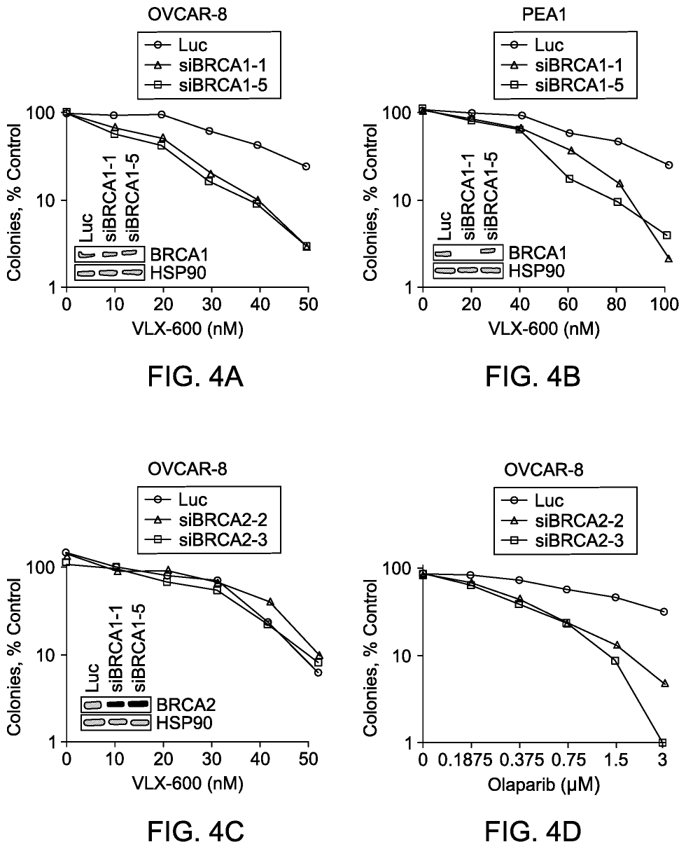

The effect of VLX600 on HR-deficient cells was next analyzed. OVCAR-8 (FIG. 4A) and PEA1 (FIG. 4B) cells transfected with 2 different BRCA1 siRNAs were far more sensitive to VLX600 than control cells (luciferase siRNA-transfected), suggesting that BRCA1 deficiency is synthetically lethal with VLX600. BRCA2 depletion, which sensitized cells to PARPi (FIG. 4D), did not sensitize to VLX600 (FIG. 4C), indicating that the effect of BRCA1 was independent of an HR defect.

### Example 4: BRCA1 Depletion Increases NNMT mRNA and Protein Expression

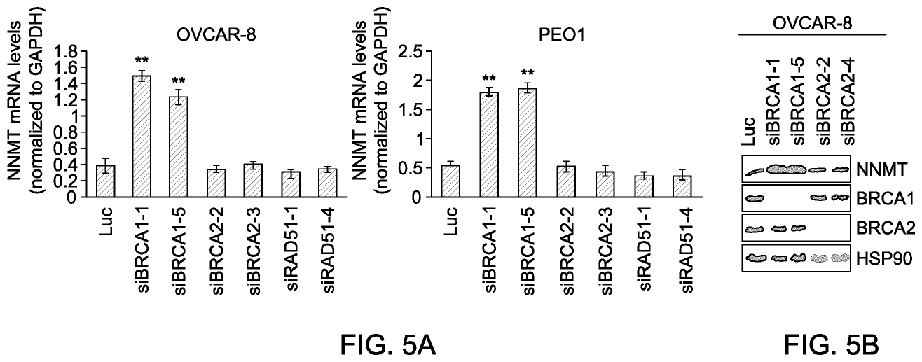

To identify how BRCA1 depletion might affect sensitivity to VLX600, RNA-seq data was examined, which showed that decreased BRCA1 levels were correlated with increased levels of NNMT, a protein that regulates mitochondrial energy metabolism. Consistent with those observations, 2 independent BRCA1 siRNAs increased NNMT mRNA and protein levels in OVCAR-8, OVCAR-5 (data not shown), and PEO1 cells (FIG. 5A,B). Depletion of the HR proteins BRCA2 and RAD51 did not affect NNMT expression, indicating that BRCA1 regulation of NNMT is independent of its role in HR.

### Example 5: Increased NNMT Caused by BRCA1 Depletion Alters Metabolism

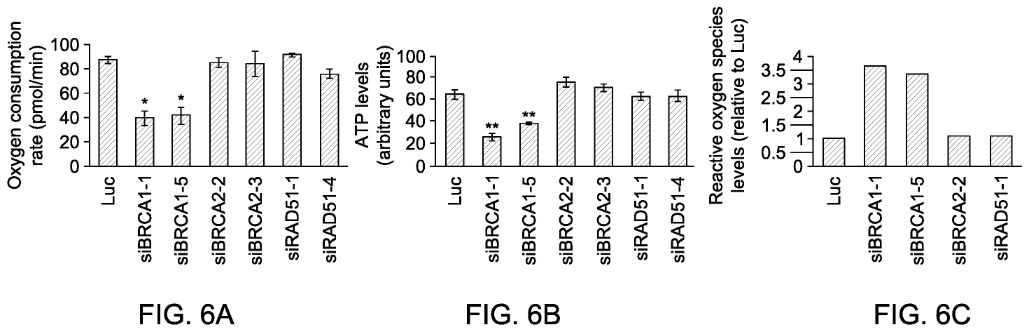

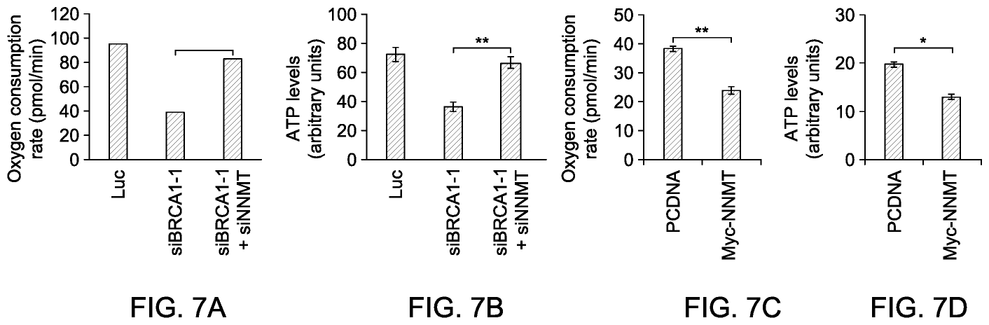

How the increase in NNMT caused by BRCA1 depletion impairs metabolism in ovarian cancer cells was evaluated. Although glycolysis was not decreased by BRCA1 depletion, BRCA1 depletion reduced the oxygen consumption rate (OCR), decreased ATP levels, and increased ROS levels (FIG. 6A-C). To determine if BRCA1 alters these metabolic phenotypes through its effect on NNMT expression, BRCA1 and NNMT were co-depleted in ovarian cancer cells and OCR and ATP production was examined. Co-depletion of BRCA1 and NNMT rescued the metabolic defects caused by BRCA1 depletion (FIG. 7A,B), thus demonstrating that the effects of BRCA1 depletion are mediated by NNMT overexpression. Moreover, overexpression of NNMT (FIG. 7 C,D) also reduced OCR and ATP levels, demonstrating that NNMT upregulation alone can reprogram metabolism.

### Example 6: BRCA1 Binds the NNMT Promoter

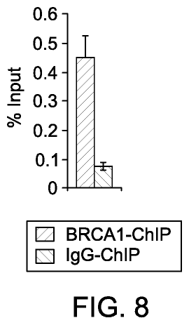

To gain insight into how BRCA1 affects NNMT transcription, chromatin immunoprecipitation (ChIP) was performed and it showed that BRCA1 interacts with the NNMT promoter (FIG. 8), suggesting that BRCA1 regulates the NNMT promoter.

### Example 7: VLX600 Synergizes with PARPi and Cisplatin in BRCA1-Depleted Cells

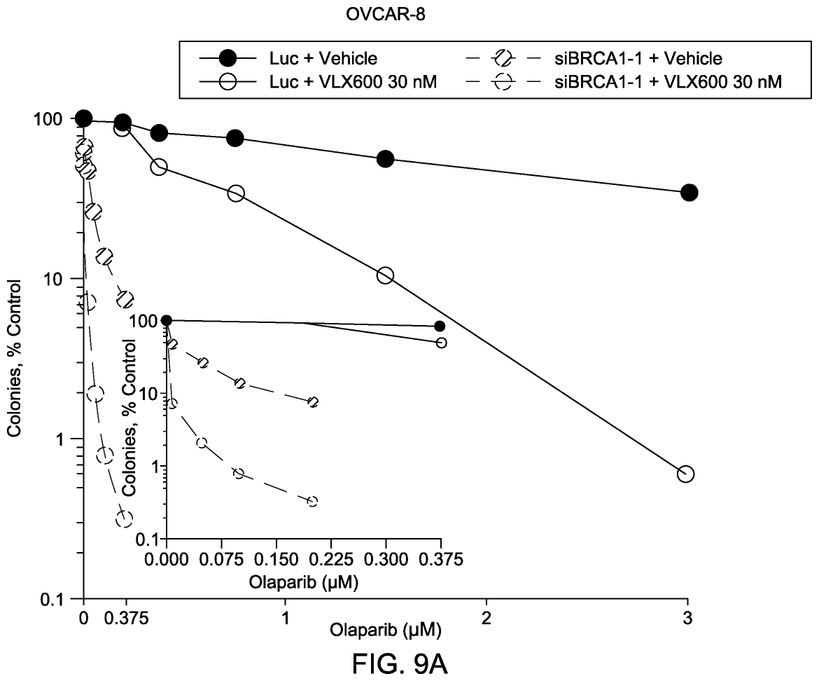

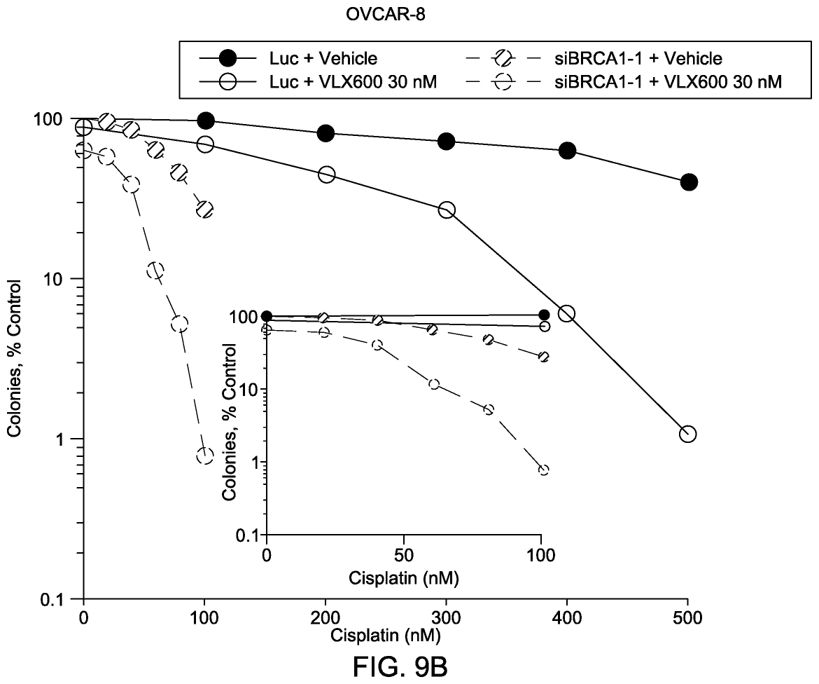

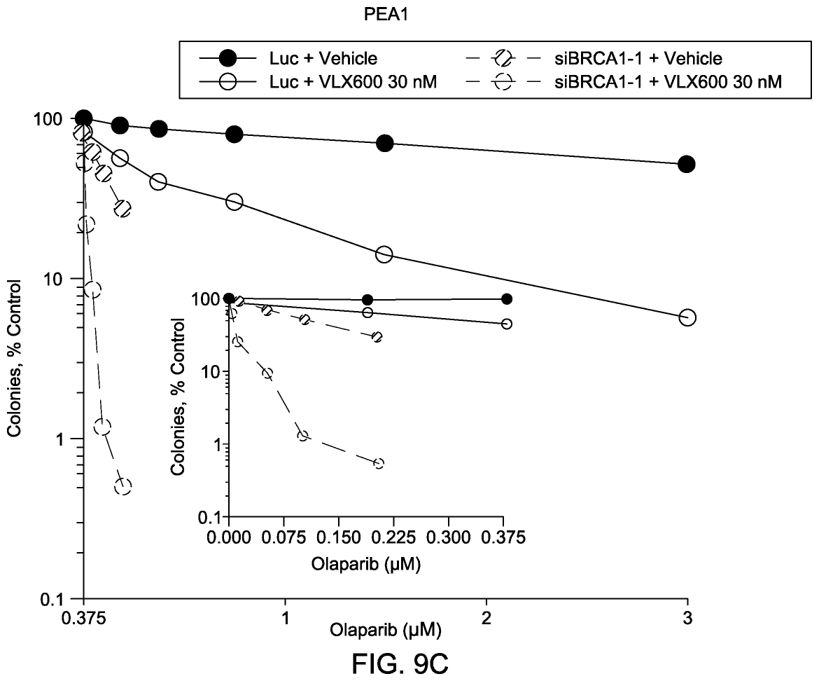

The impact of combining VLX600 with PARPi or cisplatin in BRCA1-depleted cells was next assessed. As shown in FIG. 9, VLX600 synergized with both chemotherapy agents, suggesting VLX600+PARPi (or platinum) combinations may be particularly effective in BRCA1-deficient cells.

### Example 8: BRCA1 Deficiency Upregulates NNMT, which Reprograms Metabolism and Sensitizes Ovarian Cancer Cells to Mitochondrial Metabolic Targeting Agents

This Example reports that loss of BRCA1, induced by downregulation of either BRCA1 or CDK12, impairs mitochondrial respiration and reduces ATP levels. Notably, these metabolic changes are dependent on and phenocopied by NNMT overexpression, indicating that NNMT drives the metabolic remodeling. BRCA1 depletion or NNMT overexpression confers sensitivity to agents that inhibit glucose transport and mitochondrial oxidative phosphorylation (OXPHOS), including agents that are in clinical trials as well as FDA-approved drugs that might be repurposed. Collectively, these results suggest that metabolic changes induced by BRCA1 dysfunction and NNMT overexpression might be therapeutically exploited in BRCA1-deficient or NNMT-overexpressing HGSOC.

### Materials and Methods

### Cell Lines, Cell Culture, and Metabolism-Targeting Agents

OVCAR-8 and OVCAR-5 cells were kind gifts. The PEA1 cell line was from Sigma-Aldrich. The cells were cultured in RPMI-1640 medium (Corning) supplemented with 8% fetal bovine serum (Millipore), and maintained in a humidified 37° C., 5% CO₂ incubator. All cells were authenticated by autosomal STR profiling (University of Arizona Genetics Core). VLX600 was obtained from Cayman Chemical. Tigecycline and WZB117 were obtained from Selleck Chemicals.

siRNAs and siRNA Transfection

All siRNAs were purchased from Dharmacon. siRNAs used were:

| | | |
|---|---|---|
| luciferase | 5′-CUUACGCUGAGUACUUCGA-3′ | SEQ ID NO: 1 |
| BRCA1 #1 | 5′-GUGGGUGUUGGACAGUGUA-3′ | SEQ ID NO: 2 |
| BRCA1 #2 | 5′-GAAGGAGCUUUCAUCAUUC-3′ | SEQ ID NO: 3 |
| CDK12 #1 | 5′-GAGACUAGACAAUGAGAAA-3′ | SEQ ID NO: 4 |
| CDK12 #2 | 5′-GCUGAAUAACAGUGGGCAA-3′ | SEQ ID NO: 5 |
| BRCA2 #1 | 5′-GACUCUAGGUCAAGAUUUA-3′ | SEQ ID NO: 6 |
| BRCA2 #2 | 5′-GAAGAAUGCAGGUUUAAUA-3′ | SEQ ID NO: 7 |
| RAD51 #1 | 5′-GGGAUUUGUGAAGCCAAA-3′ | SEQ ID NO: 8 |
| RAD51 #2 | 5′-CCAACGAUGUGAAGAAAUU-3′ | SEQ ID NO: 9 |

siRNA transfections (2 μM siRNA/transfection) were conducted as described elsewhere (see, e.g., Huntoon et al., Cancer Res., 73:3683-91 (2013)).

### Plasmids, Plasmid Transfection, and Stable Cell Line Generation

A mammalian expression plasmid that encoded human NNMT fused to Myc and DDK tags at its C terminus was obtained from Origene (Cat #RC200641). For the generation of stable NNMT overexpressing OVCAR-8 cell line, the NNMT-Myc-DDK plasmid or empty vector control was transfected (5 μg/transfection) into OVCAR-8 cells (8×10⁶ cells/transfection) using a BTX ECM 830 electroporator. Cells were plated in 10-cm dishes containing RPMI supplemented with 8% fetal bovine serum and incubated for 48 hours. After G418 (2 mg/mL) was added, the cells were cultured for an additional 12 days, replenishing the selection medium every 3 days. The resistant clones were trypsinized using 0.25% Trypsin-EDTA (Life Technologies) and reseeded at 50 cells per dish into 15-cm dishes containing 2 mg/mL G418. After 10 days of culture, single colony clones were picked, expanded in 24-well plates containing complete medium plus G418. Seven to 10 days later, 10 empty vector or NNMT cell clones were isolated and assayed for NNMT by immunoblotting for each stably transfected cell line. To generate the SFB-BRCA1 mammalian expression plasmid, human full-length BRCA1 cDNA was subcloned into the pSFB vector that contains in-frame N-terminal S-peptide, FLAG, and streptavidin-binding peptide tags.

### Immunoblotting

Immunoblotting was performed as described elsewhere (see, e.g., Huntoon et al., Cancer Res., 70:8642-50 (2010)). Primary antibodies used were: mouse monoclonal CDK12, which was generated in our laboratory (Clone 1.11.1 B9, 1:100); mouse monoclonal BRCA1 (1:2000, sc-6954, Santa Cruz Biotechnology); rabbit polyclonal BRCA2 (1:5000, A303-434A, Bethyl Laboratories Inc.); mouse monoclonal NNMT (1:5000, ab119758, Abcam); rabbit polyclonal RAD51 (1:2000, PC-130, Calbiochem); and mouse monoclonal HSP90 (1:1000; D. Toft, Mayo Clinic, H9010). Secondary antibodies used were: horseradish peroxidase-conjugated anti-mouse immunoglobulin G (1:10,000 for CDK12, 1:2000 for BRCA1, and 1:20,000 for NNMT and HSP90, 7076S, Cell Signaling Technology) and anti-rabbit immunoglobulin G (1:5,000 for BRCA2 and 1:10,000 for RAD51, 7074S, Cell Signaling Technology).

Quantitative Real-Time PCR (qPCR)

Total RNA isolation from cells was performed using a miRNeasy mini kit (Qiagen) following the supplier's instructions. cDNA was synthesized from 1 μg of total RNA using oligo(dT) primers and SuperScript™ III reverse transcriptase (ThermoFisher Scientific). qPCR was performed in triplicate for each sample (25 ng cDNA template in a final volume of 20 μL) on a CFX96 real-time PCR system (Bio-Rad) using iTaq Universal SYBR Green Supermix (Bio-Rad). mRNA expression was normalized to GAPDH. The qPCR primers used were:

| | | | |
|---|---|---|---|
| BRCA1 | Forward: | 5′-GCCAAGGCAAGATCTAGAGG-3′ | SEQ ID NO: 10 |
| | Reverse: | 5′-GTTGCCAACACGAGCTGA-3′ | SEQ ID NO: 11 |
| NNMT | Forward: | 5′-TGATCATGGATGCGCTCAAG-3′ | SEQ ID NO: 12 |
| | Reverse: | 5′-TTGCGAGATCACCTCAAACC-3′ | SEQ ID NO: 13 |
| GAPDH | Forward: | 5′-GAAGGTGAAGGTCGGAGTCA-3′ | SEQ ID NO: 14 |
| | Reverse: | 5′-AATGAAGGGGTCATTGATGG-3′ | SEQ ID NO: 15 |

### Clonogenic Assays

Clonogenic assays were performed as described elsewhere (see, e.g., Wagner et al., Mol. Pharmacol., 76:208-14 (2009)).

### Seahorse Assay

Twenty-four hour after siRNA transfection, OVCAR-8 or PEA1 cells were plated at 8,000 or 7,000 cells/well, respectively, onto Seahorse 8-well XFp cell culture miniplates and allowed to grow for another 24 hours before being assayed for oxygen consumption rate (OCR) and extracellular acidification rate (ECAR) on a Seahorse XFp extracellular flux analyzer (Seahorse Bioscience, Agilent Technologies). One hour prior to the start of the assay, cells were washed and changed to Seahorse XF base assay medium adjusted to pH 7.4, and incubated in a 37° C. non-CO₂ incubator. OCR was measured using the Seahorse XFp Cell Mito Stress Test Kit (Agilent Technologies) under basal conditions and in response to 1 μM oligomycin, 0.5 μM carbonyl cyanide p-trifluoromethoxyphenylhydrazone (FCCP), and 0.5 μM rotenone and antimycin A. ECAR was measured under basal conditions and after sequential injections of 10 mM glucose, 2.5 μM oligomycin and 50 mM 2-deoxy-glucose. OCR and ECAR measurements were taken at three time points before and after the addition of each inhibitor.

### ATP Assay

Forty-eight hours after siRNA transfection, total ATP levels in the cells were measured colorimetrically by using an ATP assay kit (Cat. No. ab83355, Abcam).

### RNA-Seq Analysis

Forty-eight hour after CDK12 #1 or CDK12 #2 siRNA transfection, RNA was isolated using miRNeasy mini kit (Qiagen). Three independent RNA samples were prepared for each of the transfected siRNAs. Libraries were prepared (Illumina TruSeq mRNA v2) and processed through Mayo Clinic's MAP-RSeq (v2.1.0) application, a comprehensive computational pipeline for the analysis of Illumina's paired end RNA-Sequencing reads. MAP-RSeq uses publically available bioinformatics tools tailored by in-house developed methods. Within MAP-RSeq, TopHat2 with the bowtie1 option was called to align each sample's reads to the hg19 reference genome. The first 100,000 reads of each sample were used to estimate the mean and the standard deviation of the fragment length, which is required information for TopHat. The gene counts were generated by FeatureCounts using Ensembl's hg19 gene definition file. The “−O” option within FeatureCounts was used to account for the expression derived from regions shared by multiple genomic features. RSeqQC was used to create quality control metrics, including gene body coverage plots, to insure the results from each sample were reliable and could be collectively used for a differential expression analysis. Genes with an average of less than 25 reads per group were removed from the differential expression analysis. The R package (v3.3.1), edgeR was used to identify which genes were differentially expressed across group comparisons. Statistically significant genes were defined by having a false discovery rate below 0.0001 and an absolute log 2 fold change greater than 0.75.

### Results

### CDK12 Depletion Disrupts Mitochondrial Metabolism, Reducing Respiration but not Glycolysis

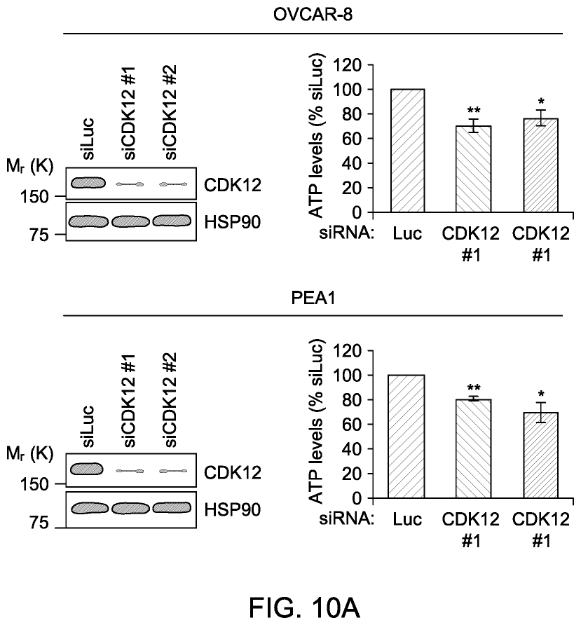

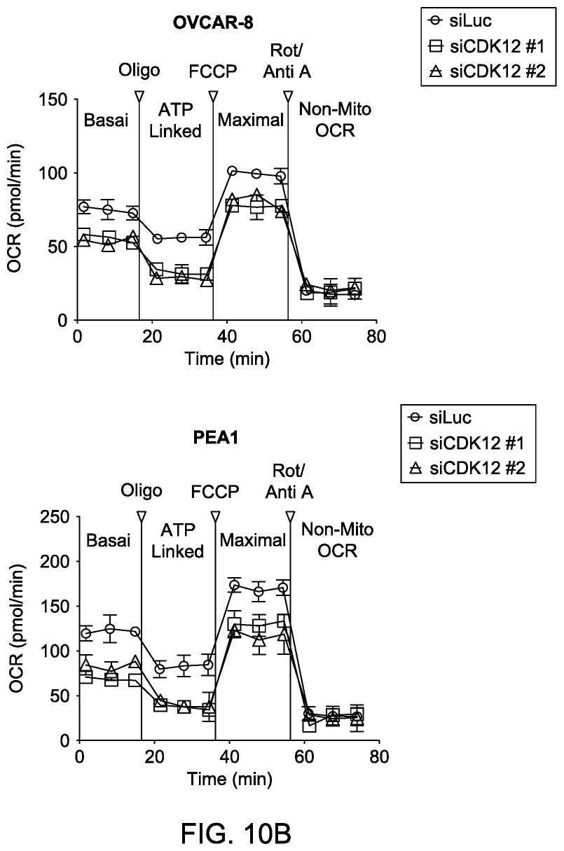

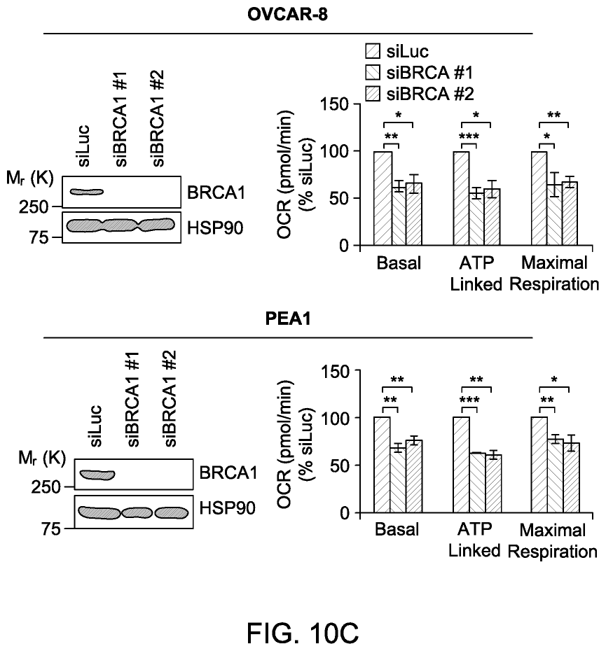

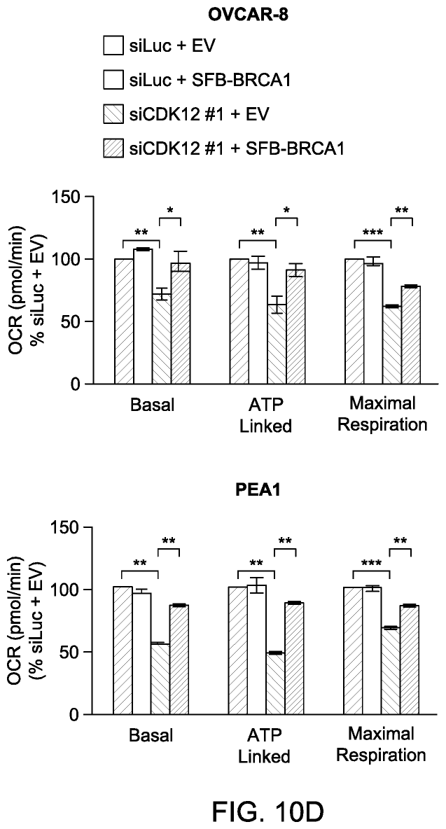

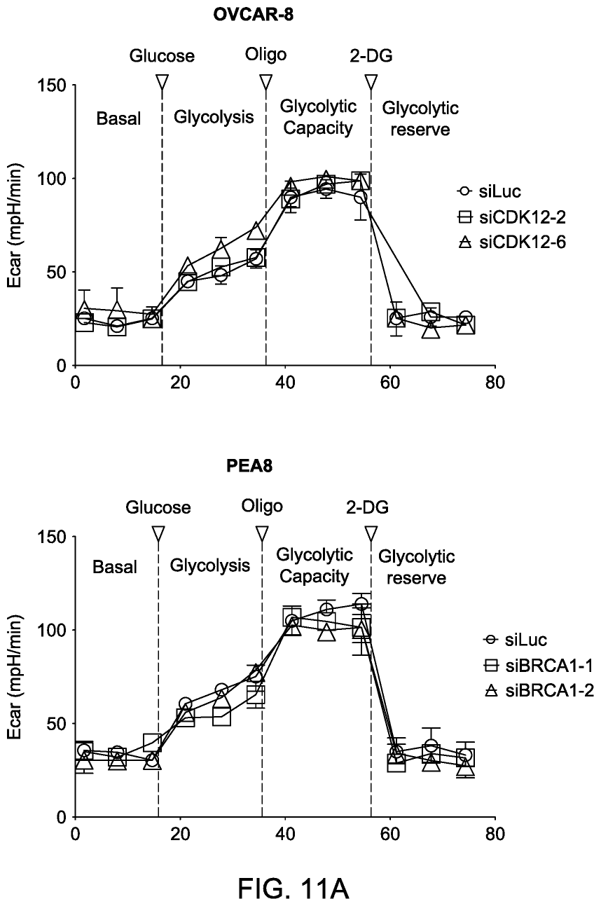

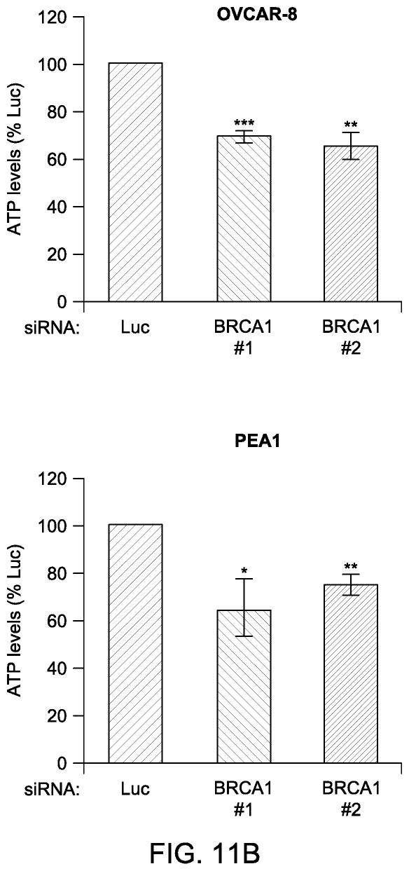

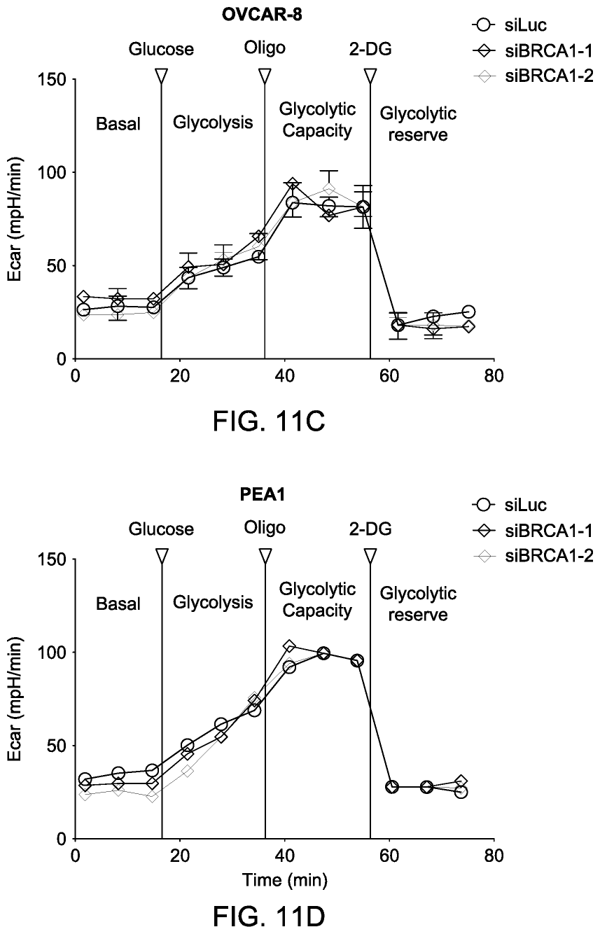

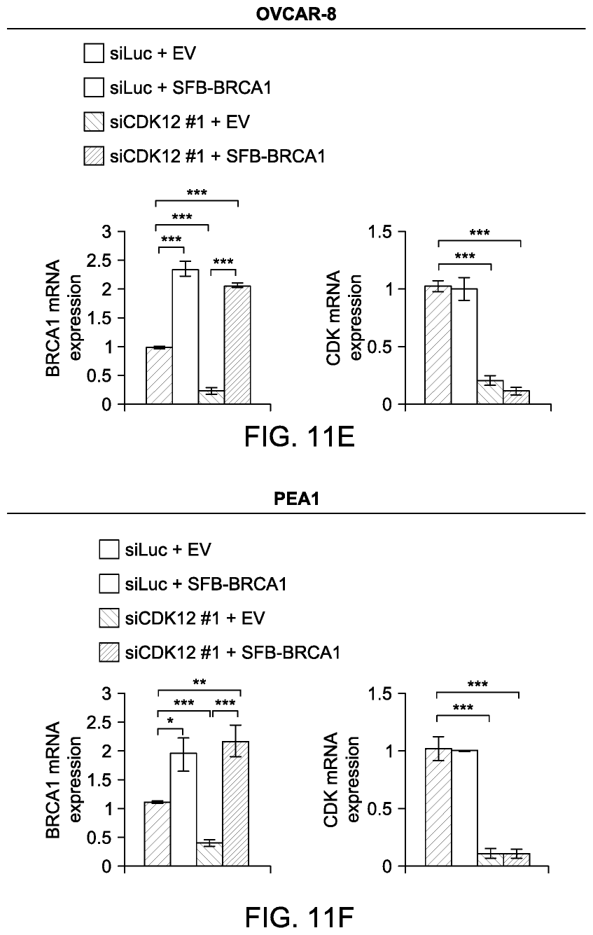

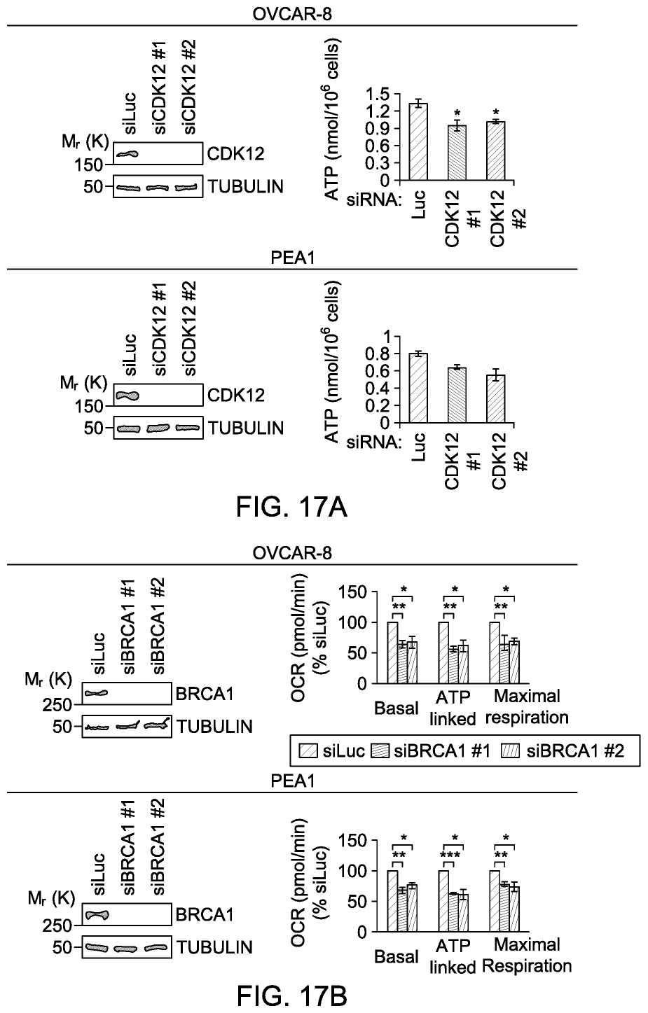

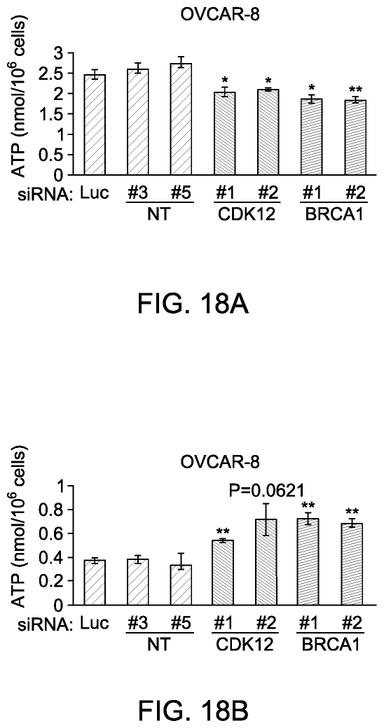

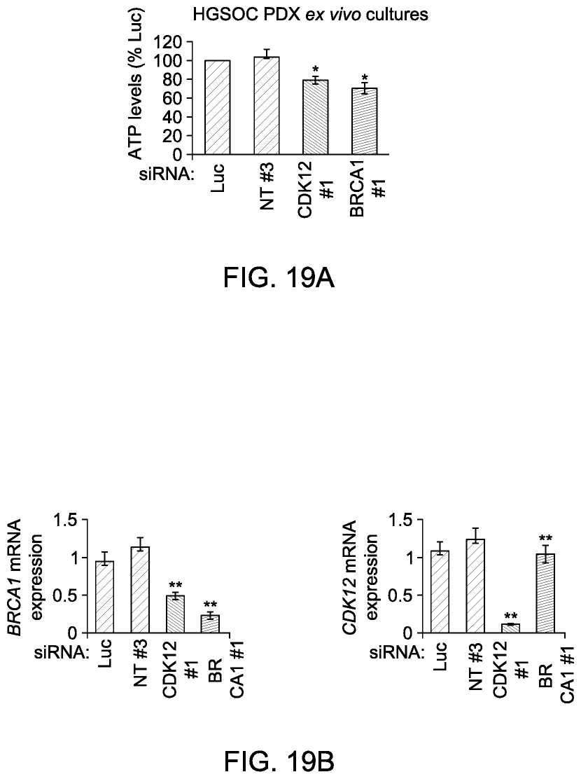

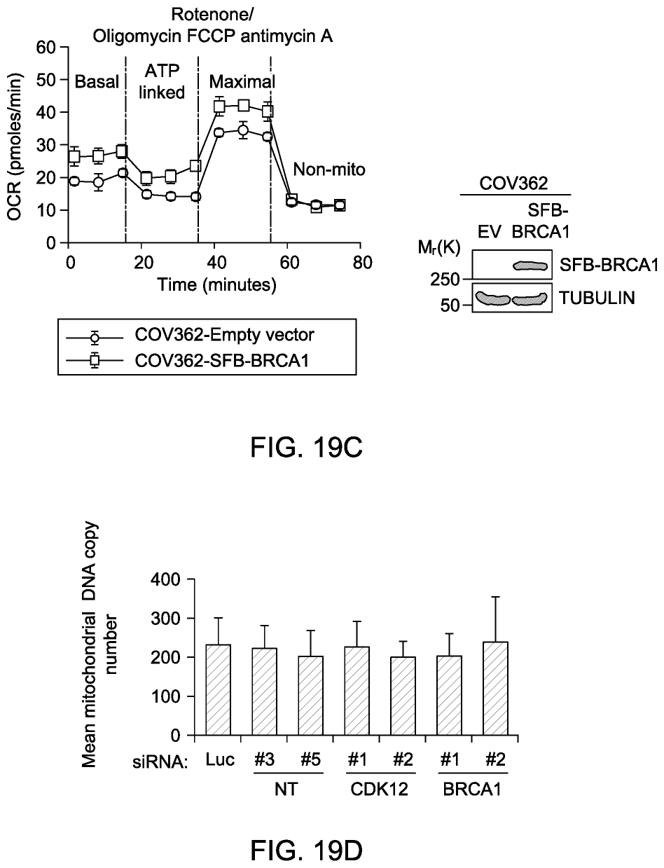

While assessing how loss of CDK12 affects ovarian cancer cells, it was observed that CDK12 depletion reduced ATP levels in OVCAR-8 and PEA1 cells (FIG. 10A and FIG. 17A). Because ATP is produced primarily by OXPHOS or glycolysis, the metabolic activity of these cells was profiled using extracellular flux analyses to determine whether either process was affected by CDK12 depletion. These studies showed that the extracellular acidification rate (ECAR), an indicator of glycolysis, was not reduced by CDK12 depletion (FIG. 11A). In contrast, the oxygen consumption rate (OCR), an indicator of OXPHOS, was reduced under basal conditions (FIG. 10B) as well as after treatment with oligomycin, an inhibitor of ATP synthase; carbonyl cyanide p-trifluoromethoxyphenylhydrazone (FCCP), an uncoupler of mitochondrial oxidative phosphorylation; and rotenone and antimycin A, two inhibitors of mitochondrial electron transport complexes I and II, respectively. These findings demonstrate that CDK12 depletion reprograms mitochondrial metabolism, leading to reduced OCR and ATP production.

### Metabolic Reprogramming Induced by CDK12 Depletion is Phenocopied by BRCA1 Depletion

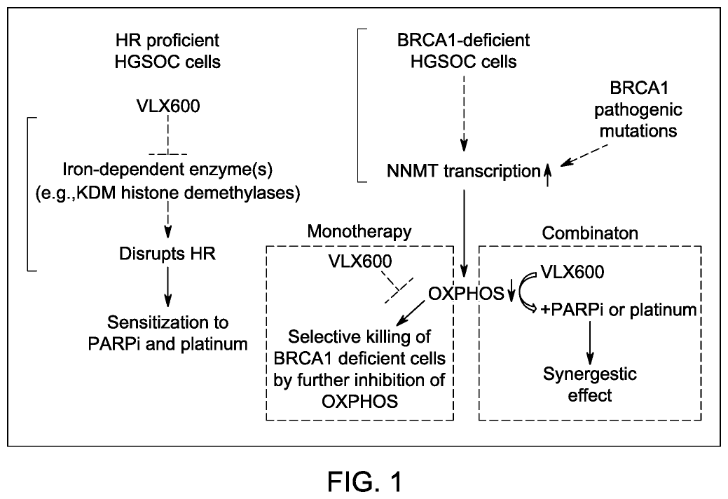

Whether the mitochondrial respiration defects induced by CDK12 loss may actually be caused by loss of BRCA1 was examined. Consistent with this possibility, BRCA1 depletion also reduced OCR (FIG. 10C and FIG. 17B) and ATP production (FIG. 11B) in these cell lines without disrupting glycolysis (FIGS. 11C and D). Moreover, re-expression of exogenous BRCA1 restored the OCR in CDK12-depleted cells (FIG. 1D; FIGS. 11E and F), thus demonstrating that reduced OCR caused by CDK12 depletion was driven by loss of BRCA1.

Disabling HR does not Cause the Mitochondrial Dysfunction Induced by BRCA1 Depletion

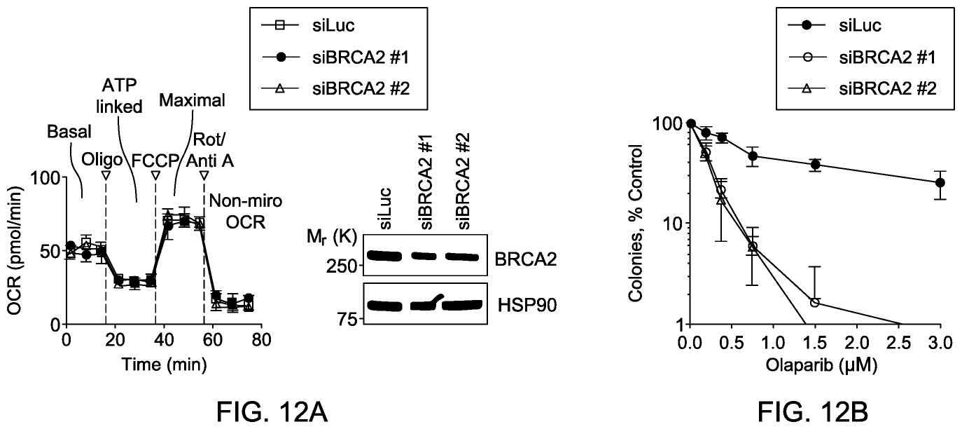

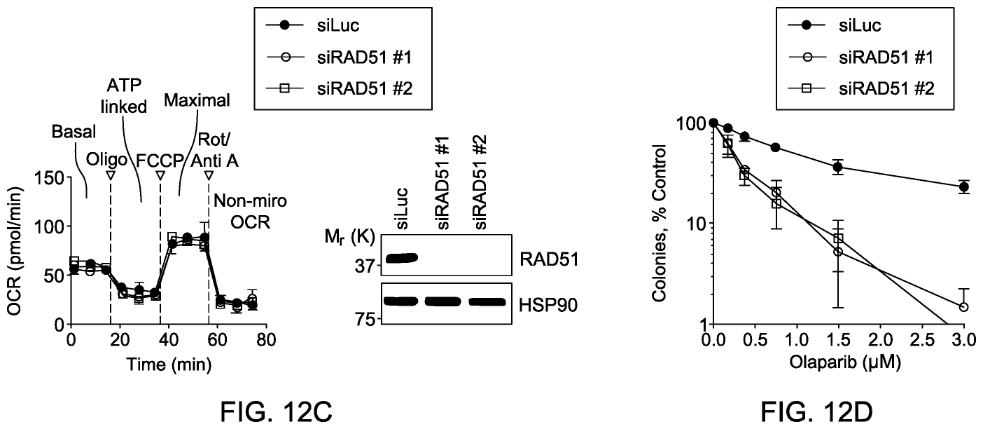

Whether an HR defect induced by loss of other HR proteins would similarly cause the mitochondrial defect caused by BRCA1 depletion was examined. In contrast to BRCA1 depletion, depletion of BRCA2 and RAD51, two key participants in HR, did not cause defects in OCR (FIGS. 12A and C) but did disrupt HR, as shown by increased sensitivity to the PARPi olaparib (FIGS. 12B and D). Taken together, these results suggest that the role of BRCA1 in metabolism is independent of its role in HR and support a model in which BRCA1 deficiency reprograms mitochondrial metabolism in ovarian cancer.

### Upregulation of NNMT Drives the Metabolic Alteration Caused by BRCA1 Depletion

To gain insight into how CDK12 and BRCA1 depletion affected mitochondrial respiration, an RNA-seq dataset from control and from CDK12-depleted OVCAR-8 cells was analyzed. CDK12 depletion altered the expression of genes involved in DNA replication, DNA repair, RNA processing, and RNA splicing. However, NNMT mRNA levels were increased ˜4 fold in the CDK12-depleted cells.

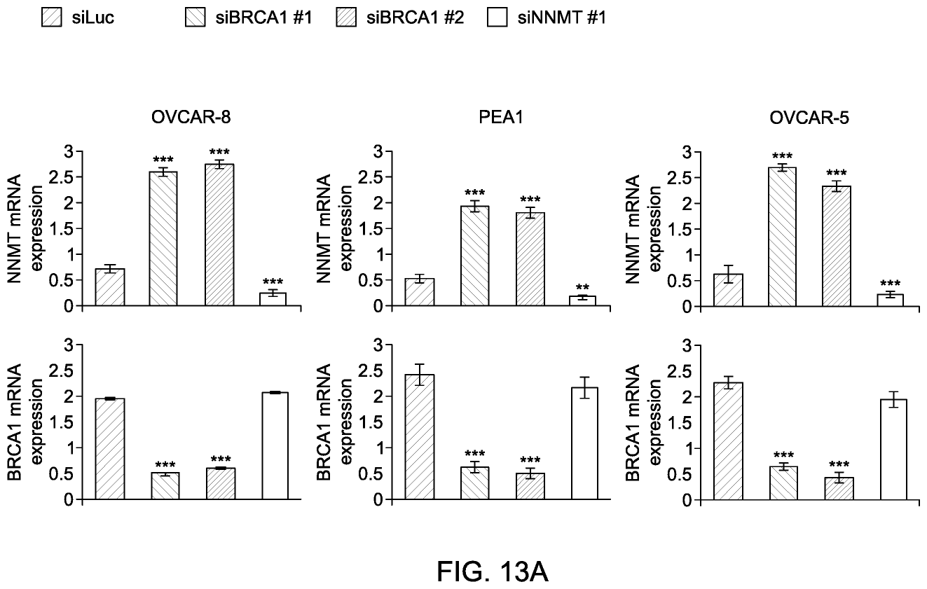

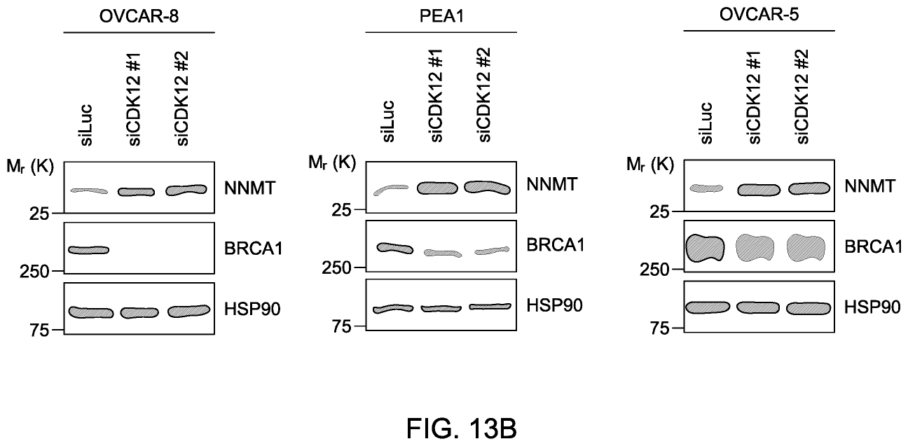

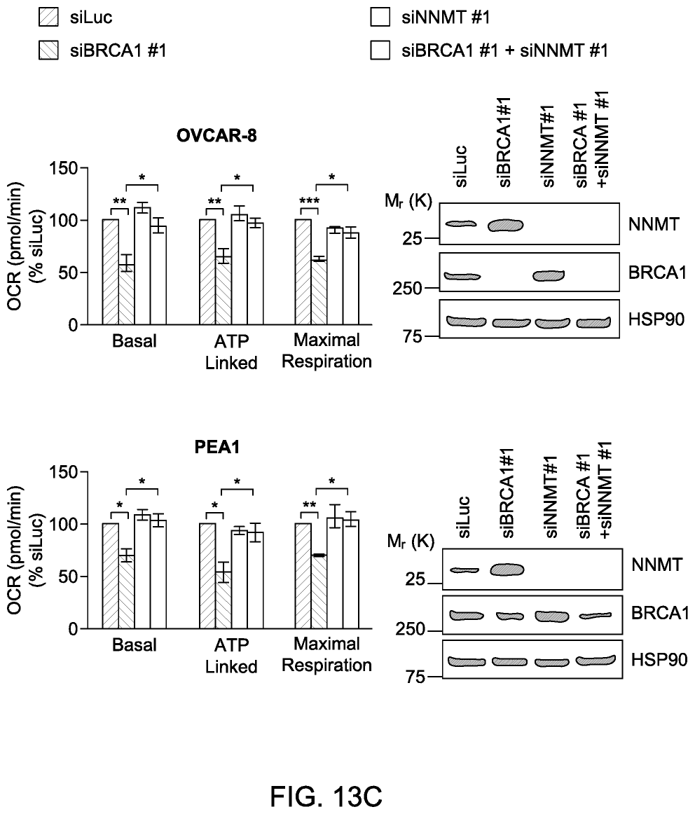

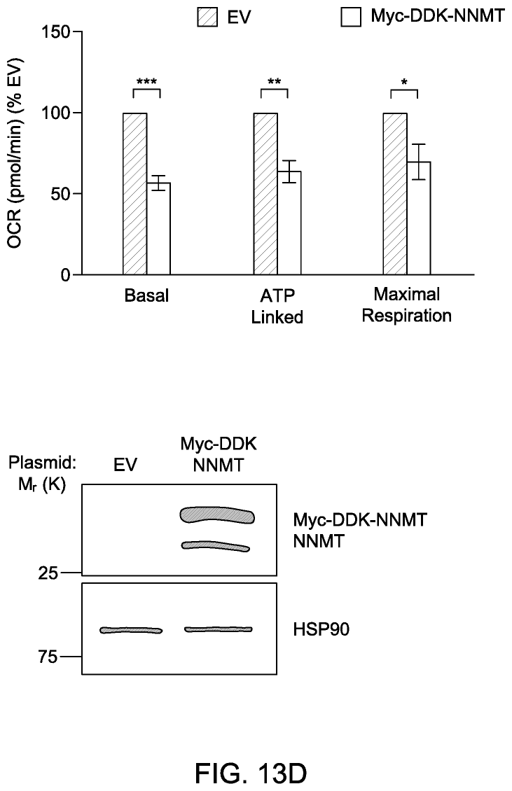

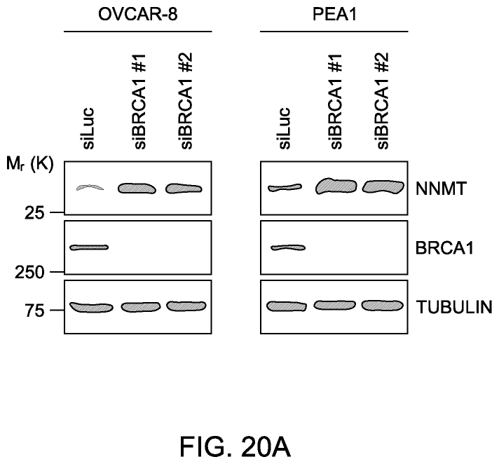

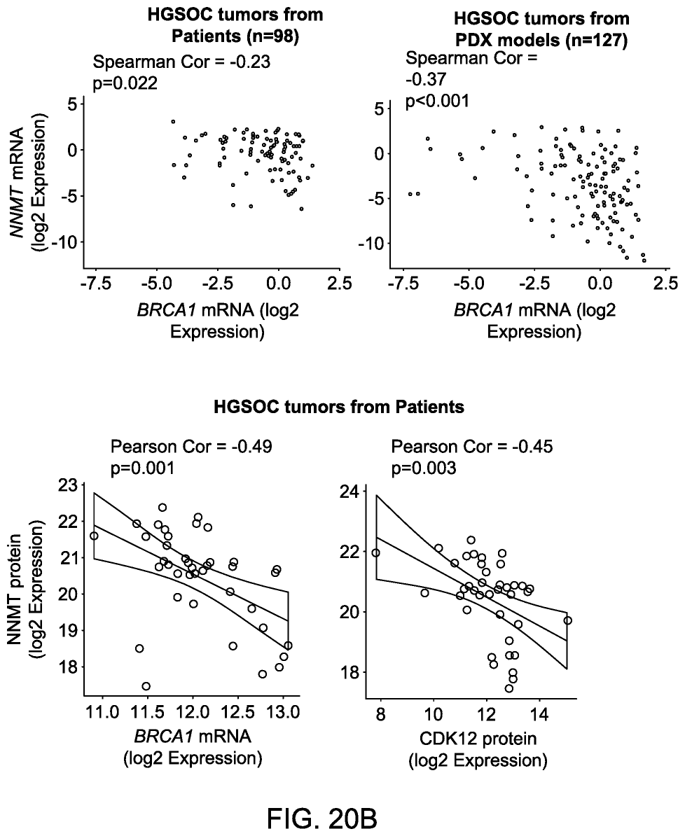

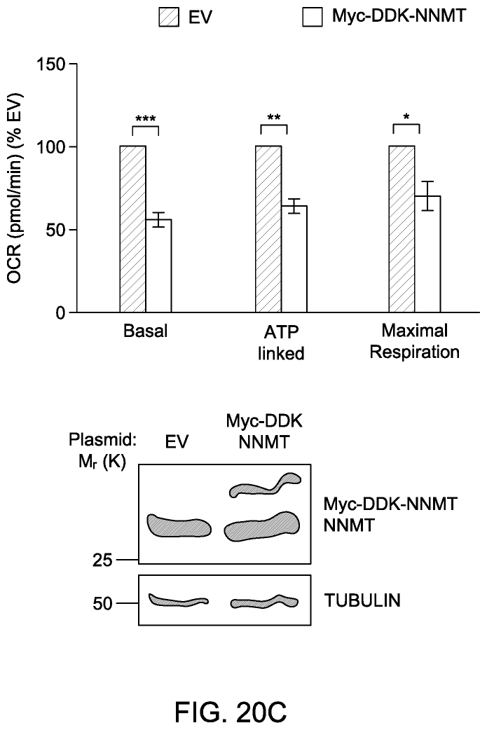

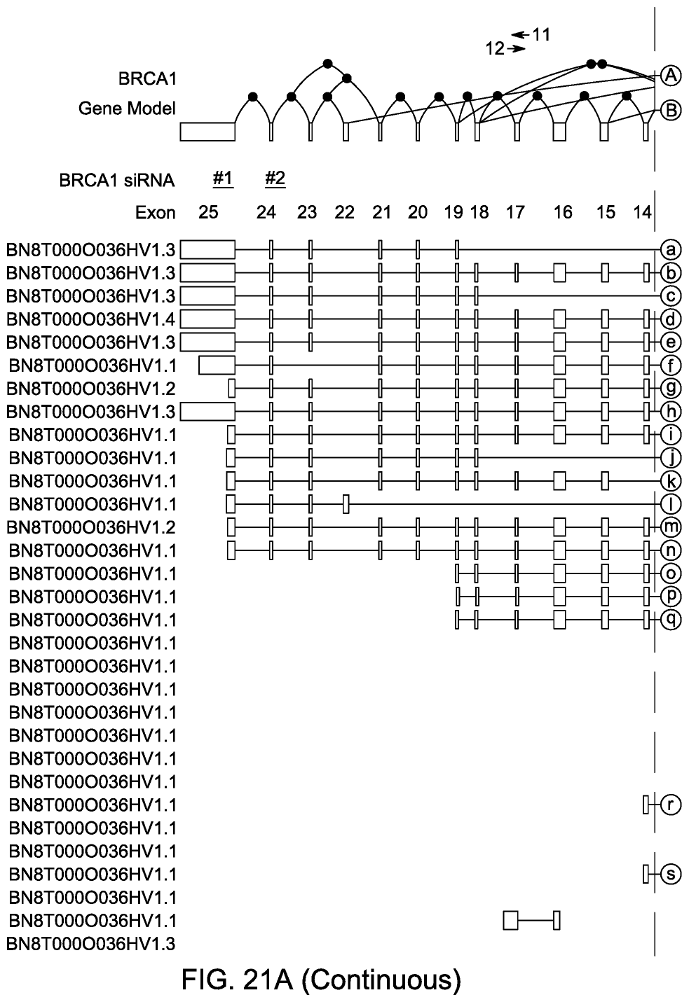

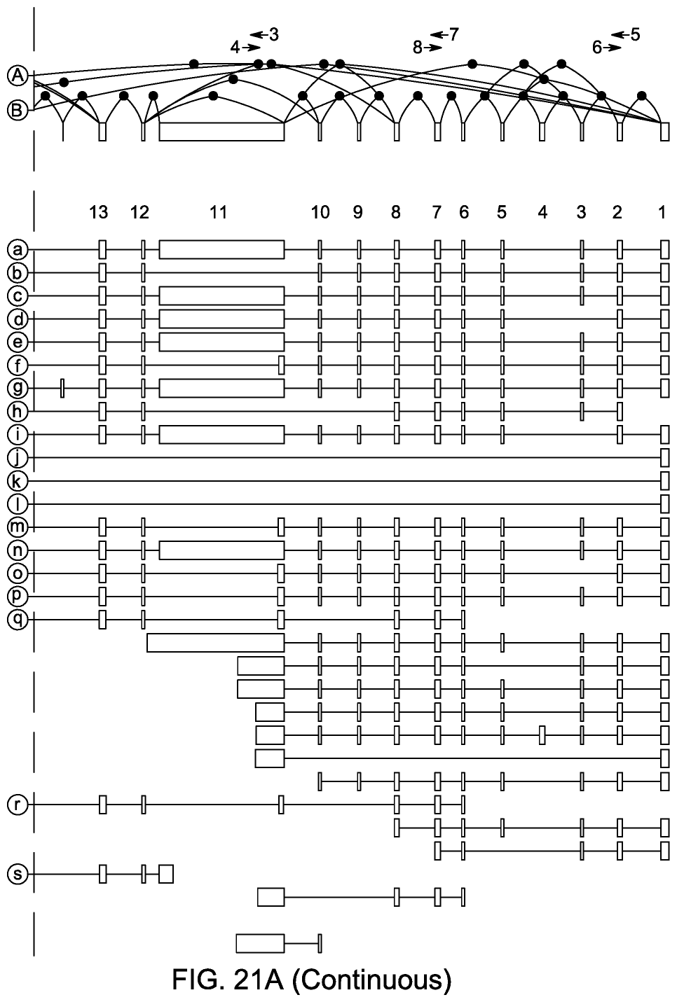

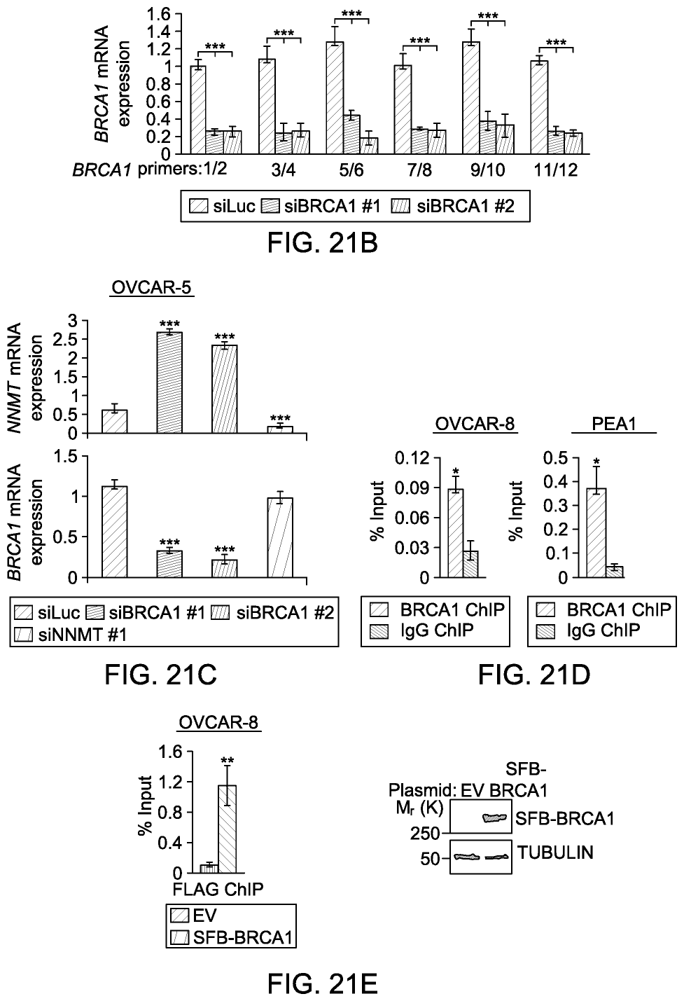

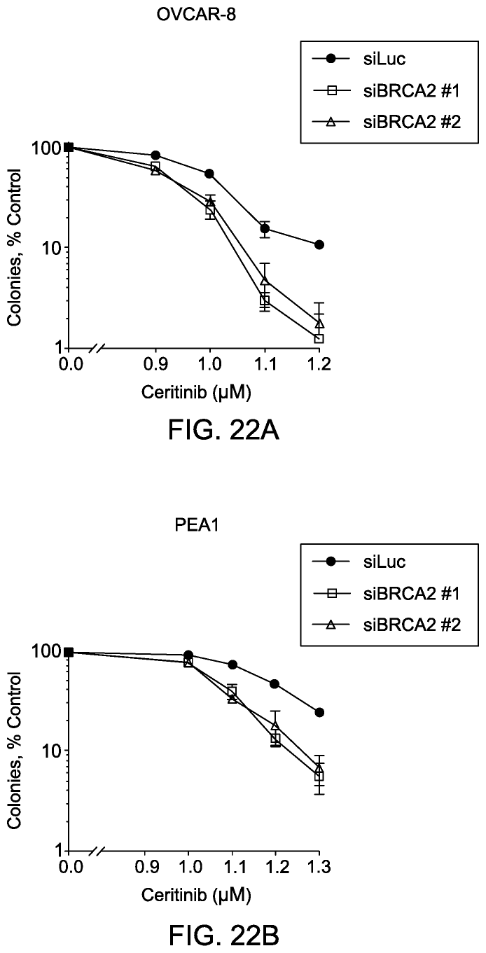

Whether NNMT overexpression could drive the decrease in OCR was examined. To explore this possibility, it was first demonstrated that two independent BRCA1 siRNAs increased NNMT mRNA (FIG. 13A) and protein (FIG. 13B and FIG. 20A) levels in multiple ovarian cancer cell lines, thus validating the RNA-seq results. Second, to determine if BRCA1 drives these metabolic effects by upregulating NNMT, NNMT and BRCA1 were co-depleted, and it was found that NNMT depletion reversed the OCR defect caused by BRCA1 depletion (FIG. 13C). Finally, it was observed that transient overexpression of NNMT reduced OCR (FIG. 13D and FIG. 20C). Taken together, these results demonstrate that NNMT is necessary and sufficient to reprogram mitochondrial metabolism in ovarian cancer cells.

### BRCA1 Depletion Sensitizes Ovarian Cancer Cells to Mitochondrial Metabolic Targeting Agents

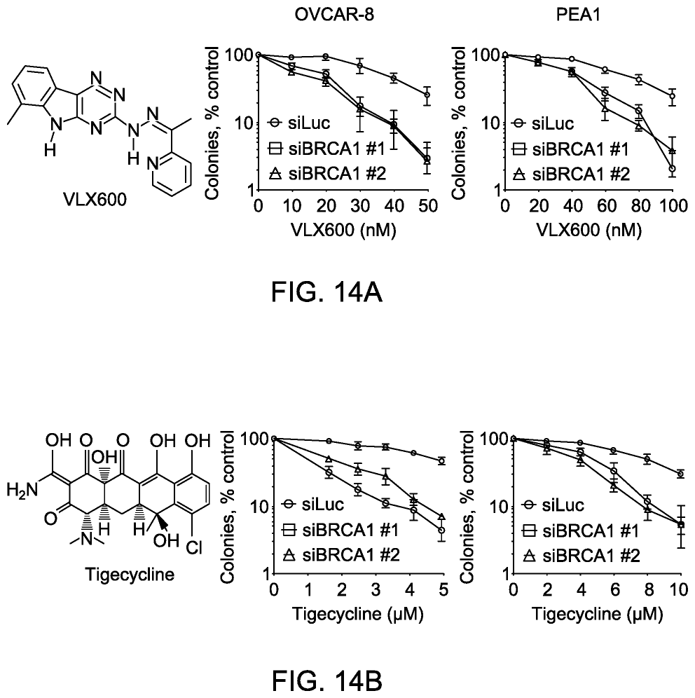

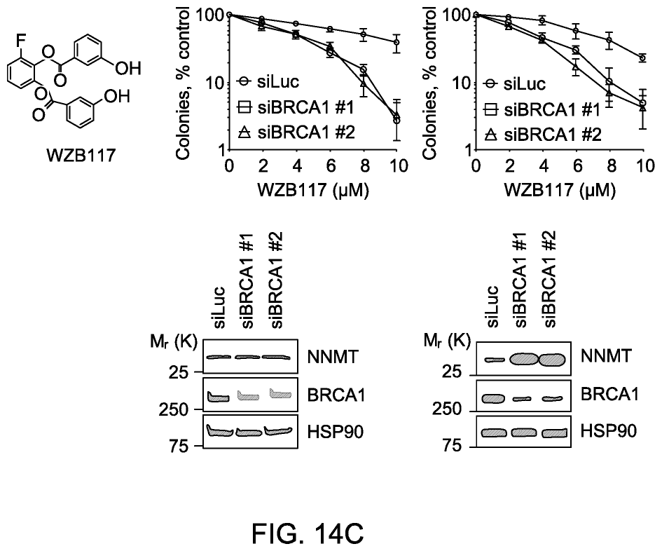

Because BRCA1 depletion disrupted mitochondrial respiration and reduced ATP levels (FIG. 10C and FIG. 17B; FIG. 11B), it was reasoned that BRCA1-deficient cells would be more sensitive to agents that cause additional metabolic stress. To test this possibility, the effect of several small molecules that inhibit energy metabolism was assessed. These include: 1) VLX600, an iron chelator that targets metabolically compromised tumors by causing mitochondrial dysfunction and inhibiting mitochondrial respiration; 2) tigecycline, an FDA-approved antibiotic that inhibits mitochondrial protein translation; and 3) WZB117, which inhibits the glucose transporter GLUT1 and reduces intracellular ATP levels. As shown in FIG. 14, BRCA1 depletion upregulated NNMT and sensitized OVCAR-8 and PEA1 cells to all three agents. Consistent with the observation that loss of BRCA2 and RAD51 did not disrupt OCR (FIGS. 12A and C), depletion of these HR proteins did not sensitize cells to OXPHOS inhibition (but did sensitize to the PARP inhibitor olaparib) (FIGS. 15A and B). These findings demonstrate that the metabolic reprogramming induced by BRCA1 loss sensitizes ovarian cancer cells to agents that disrupt energy metabolism and further indicate that BRCA1 regulation of metabolism is unrelated to its role in HR.

### NNMT Overexpression Sensitizes Ovarian Cancer Cells to Mitochondrial Metabolic Targeting Agents

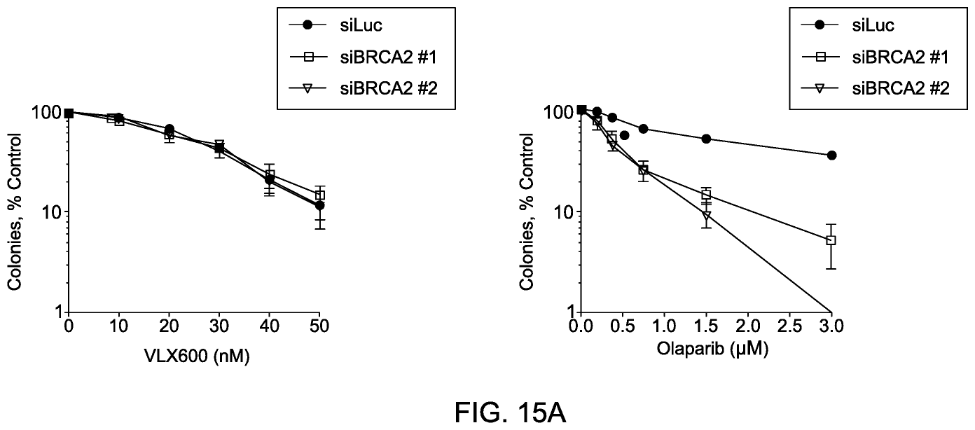

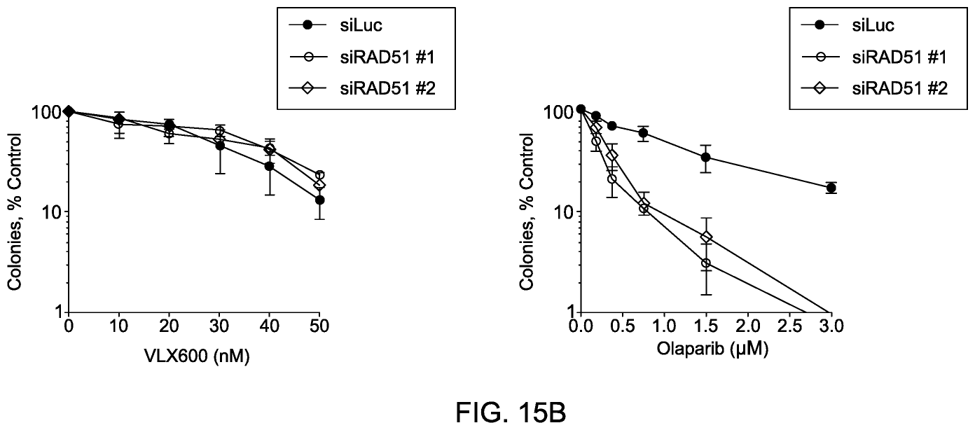

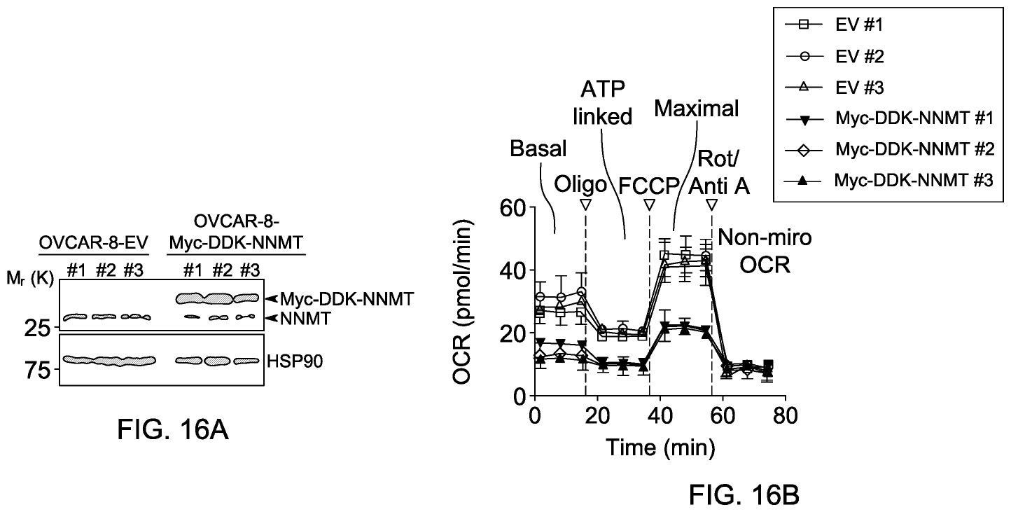

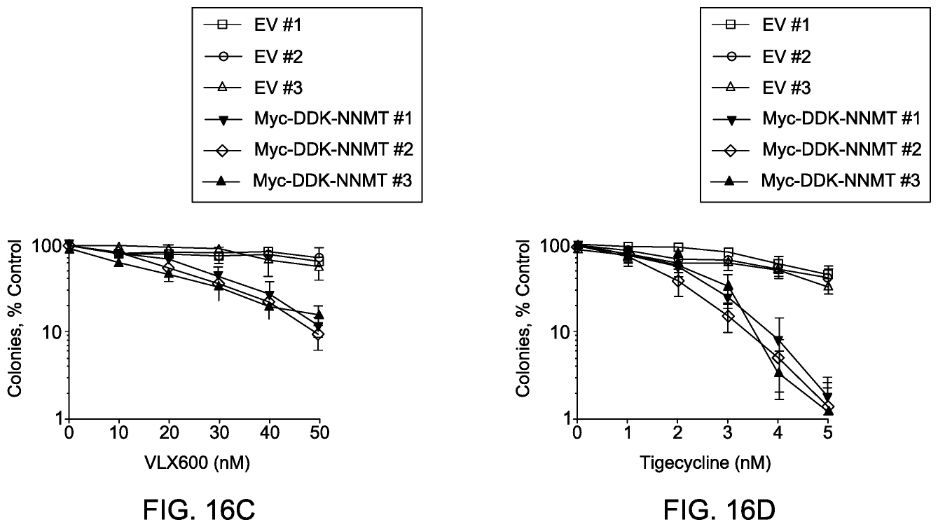

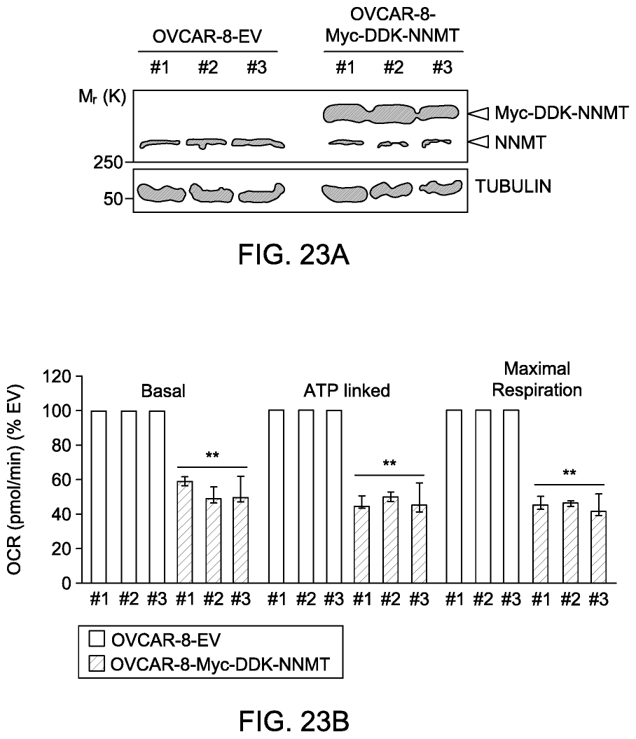

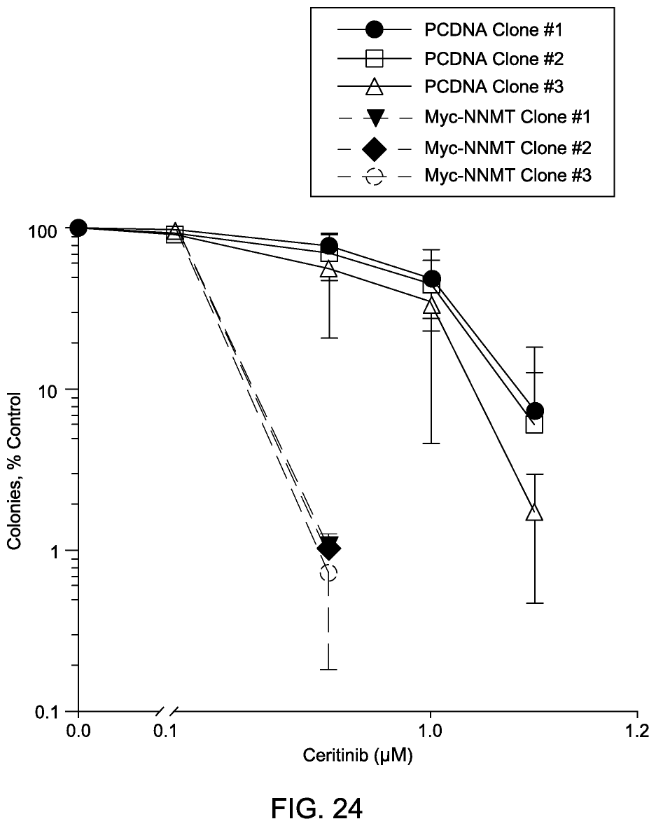

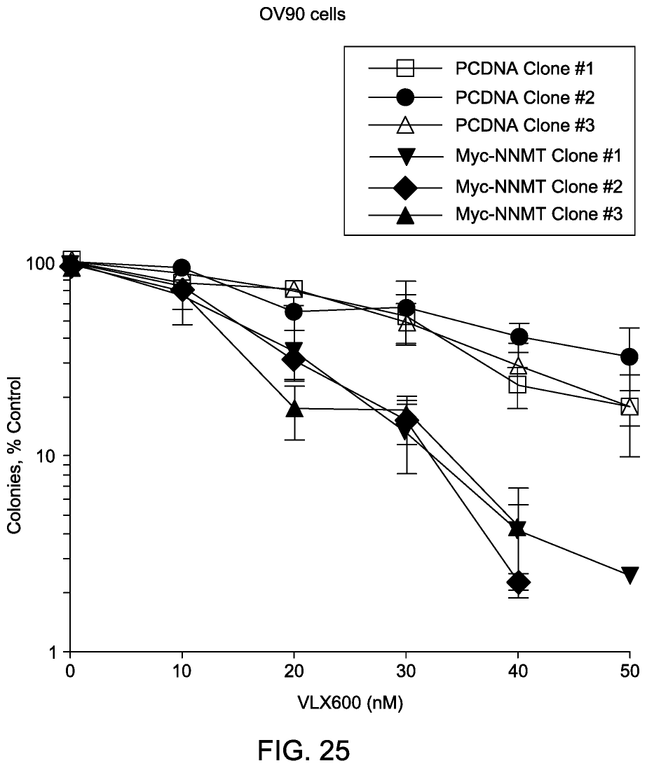

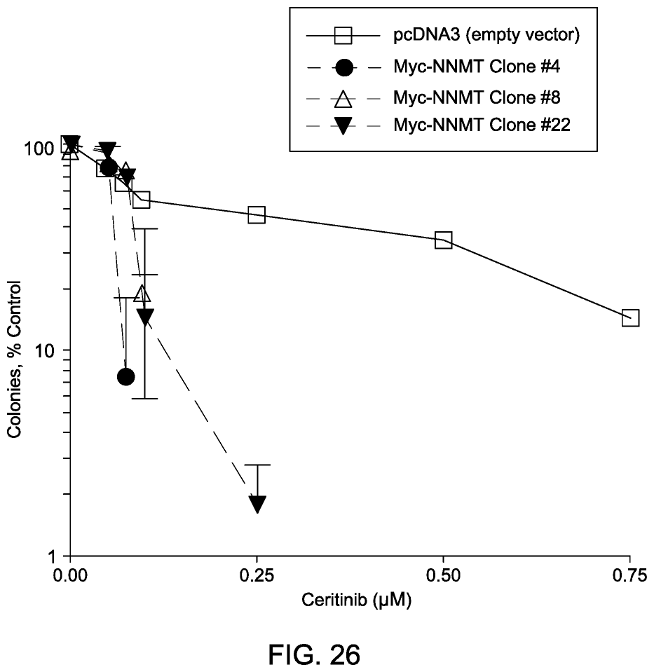

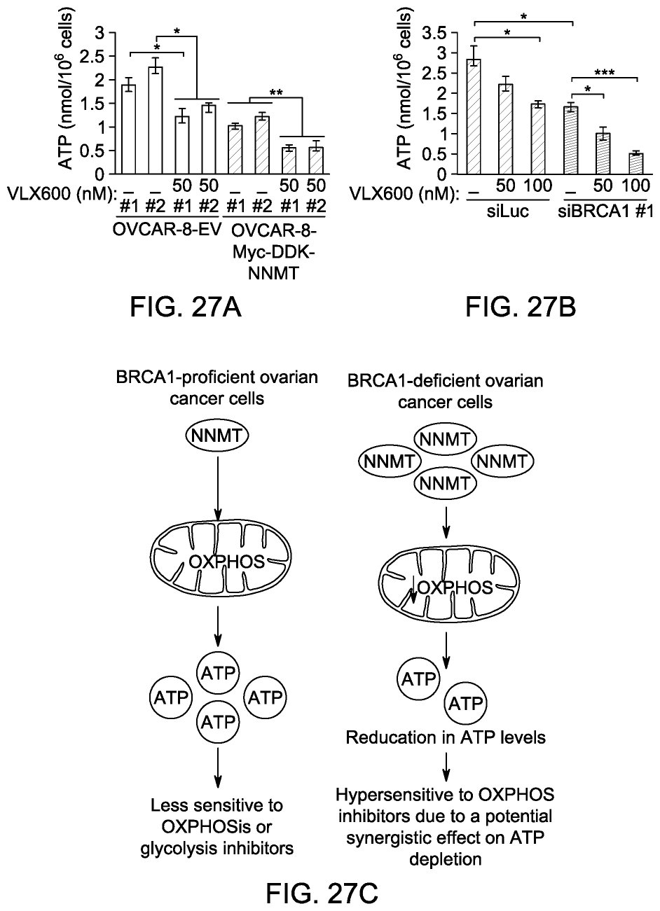

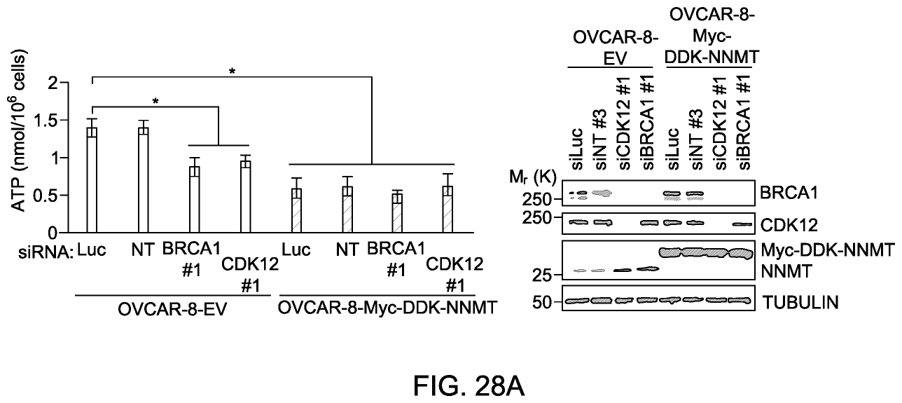

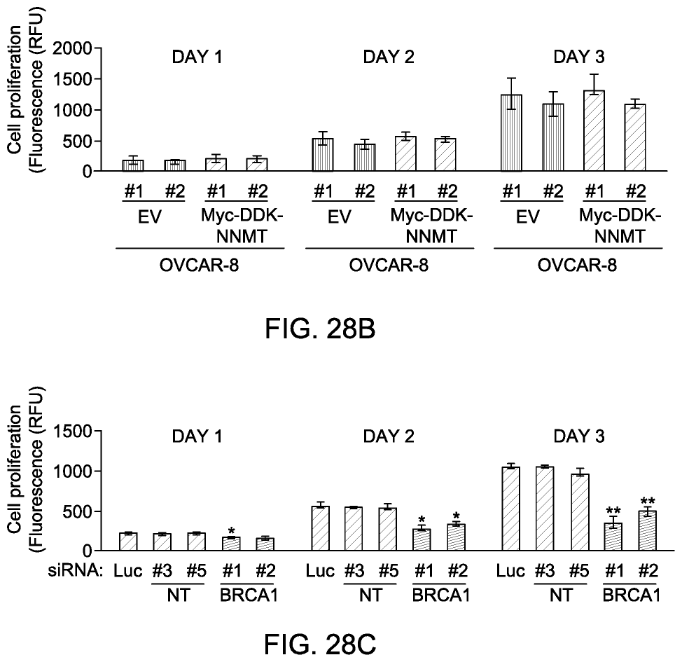

Because mitochondrial metabolic defects induced by BRCA1 depletion occurred through NNMT upregulation (FIG. 13), it was next asked whether NNMT overexpression was sufficient to sensitize ovarian cancer cells to mitochondrial metabolic targeting agents. To assess this possibility, multiple clones of OVCAR-8 cells that stably overexpress NNMT were created (FIG. 16A and FIG. 23A). As was seen with BRCA1-depletion, NNMT overexpression reduced OCR and sensitized the cells to the mitochondrial metabolic targeting agents (FIG. 16B-D), demonstrating that NNMT upregulation is sufficient to reprogram metabolism and sensitize ovarian cancer cells to agents that target mitochondrial metabolism.

Taken together, these results demonstrate that BRCA1 deficiency, via its ability to upregulate NNMT, reprograms metabolism by reducing mitochondrial respiration and sensitizes ovarian cancer cells to small molecules that disrupt energy metabolism. These results also demonstrate that BRCA1 depletion in ovarian cancer cell lines sensitized them to VLX600, tigecycline, and WZB117. In addition, these results demonstrate that cancer having BRCA1 deficiencies can be therapeutically targeted by small molecule energy metabolism inhibitors such as VLX600, tigecycline, and WZB117.

### Example 9: BRCA1 Deficiency Upregulates NNMT, which Reprograms Metabolism and Sensitizes Ovarian Cancer Cells to Mitochondrial Metabolic Targeting Agents

### Materials and Methods

### Cell Lines, Cell Culture, and Metabolism-Targeting Agent

The BRCA1-mutant COV362 cell line was a gift. The cells were cultured in RPMI-1640 medium (Corning) supplemented with 8% fetal bovine serum (Millipore), and maintained in a humidified 37° C., 5% CO₂ incubator. Ceritinib was obtained from Cayman Chemical or Selleck Chemicals.

### Generation of Stable Cell Lines

For the generation of stable NNMT overexpressing OV90 cell lines, the NNMT-Myc-DDK plasmid or empty vector control was transfected (5 μg/transfection) into OV90 cells (8×10⁶ cells/transfection) using a BTX ECM 830 electroporator (using a 4-mm cuvette with two, 280-volt, 10-msec pulses). Cells were plated in 10-cm dishes containing RPMI supplemented with 8% fetal bovine serum and incubated for 48 hours. After G418 (2 mg/mL) was added, the cells were cultured for an additional 12 days, replenishing the selection medium every 3 days. G418-resistant clones were trypsinized using 0.25% Trypsin-EDTA (Life Technologies) and reseeded at 50 cells per dish into 15-cm dishes containing 2 mg/mL G418. After 10 days of culture, single colony clones were picked, expanded in 24-well plates containing complete medium plus G418. Seven to 10 days later, 10 empty vector or NNMT cell clones were isolated and assayed for NNMT by immunoblotting for each stably transfected cell line. To generate the SFB-BRCA1 mammalian expression plasmid, human full-length BRCA1 cDNA was subcloned into the pSFB vector that contains in-frame N-terminal S-peptide, FLAG, and streptavidin-binding peptide tags. For the generation of stable BRCA1 overexpressing COV362 cells, the SFB-BRCA1 plasmid or empty vector control plasmid was transfected (40 μg/transfection) into COV362 cells (10×10⁶ cells/transfection), and after 48 hours, the cells were cultured for an additional 21 days with G418 (1 mg/mL), replenishing the selection medium every 3 days. The G418-resistant cells were used for subsequent experiments.

### Cell Proliferation Assay

Cell proliferation was assessed with the CyQUANT® Cell Proliferation Assay kit (ThermoFisher Scientific). siRNA-transfected OVCAR-8 cells or stable OVCAR-8-EV or OVCAR-8-Myc-DDK-NNMT cells were plated at 4000 cells/well in flat-bottom 96-well plates, and analyzed 24, 48, and 72 hours after plating following the supplier's instructions.

### Chromatin Immunoprecipitation (ChIP) Assays

Cells (1×10⁷) in 15-cm dishes were cross-linked with 1% formaldehyde in media for 10 minutes at room temperature, and the unreacted formaldehyde was quenched by adding 1/10 volume 1.25 M glycine (pH 7.0). The cells were harvested by trypsinization, washed with PBS, and re-suspended in cell lysis buffer (10 mM Tris, HCl, pH 7.5, 10 mM NaCl, 0.5% NP-40). After incubation on ice for 15 minutes, the chromatin fraction (pellet) was collected by centrifugation at 800×g for 5 minutes at 4° C., digested with micrococcal nuclease (2.5 units/mL; New England Biolabs) for 20 minutes at 37° C., and sonicated for 15 minutes. Aliquots of sheared chromatin were immunoprecipitated using protein G Dynabeads™ and 2 μg of mouse monoclonal BRCA1 (sc-6954, Santa Cruz Biotechnology) or mouse monoclonal FLAG (F1804, Sigma) antibodies. Normal mouse IgG (2 μg/ChIP, 0107-01, SouthernBiotech) was used as negative control. After immunoprecipitation, crosslinks were reversed by heating to 60° C., and immunoprecipitated DNA was purified using spin columns (Cat. No. 11732676001, Roche). qPCR analysis of the immunoprecipitated and genomic input DNAs was performed using iQ™ SYBR® Green Supermix (Bio-Rad). The following primers that amplify the NNMT promoter region were used: forward, 5′-CACTGCCTGTCTCTGACCAA-3′ (SEQ ID NO:16) and reverse, 5′-CAGGAGAACAGGGCTGAAAG-3′ (SEQ ID NO:17).

### Mitochondrial DNA Copy Number Analysis

OVCAR-8 cells (8×10⁶) were transfected with non-targeting control, BRCA1, or CDK12 siRNAs. Two days after transfection, genomic DNA was extracted using the Wizard® SV Genomic DNA Purification System (Promega). The relative number of copies of mitochondrial DNA were measured using the human mitochondrial DNA monitoring primer set (Cat. No. 7246; Takara) and normalized using nuclear DNA content following the supplier's protocol.

### ATP and ADP Assays

Forty-eight hour after siRNA transfection, total ATP and ADP levels in the cells were measured using colorimetric ATP assay (Cat. No. ab83355, Abcam) and ADP Assay (Cat. No. ab83359, Abcam) kits.

### HGSOC Tumor Tissues

All HGSOC tumor tissues that were used in the ex vivo culture studies, mRNA expression studies, and protein expression studies were obtained in accord with the U.S. Common Rule after written informed consent was obtained.

Ex Vivo Culture of HGSOC Tumor Tissues from PDX Mouse Models

For short-term ex vivo monolayer cultures of tumor cells, HGSOC tissues from PDX mouse models were harvested, minced into 2-4-mm pieces, and dissociated using a Tumor Dissociation Kit (Cat. #130-096-730, Miltenyi Biotec) following the supplier's protocol. The dissociated cells were washed 5 times with RPMI-1640 medium (Invitrogen) supplemented with 10% fetal bovine serum (Invitrogen), 100 units/mL penicillin, and 100 units/mL streptomycin (Invitrogen). After resuspending in RPMI-1640 medium with 10% fetal bovine serum without antibiotics, the cells were electroporated with control luciferase (Luc), non-targeting siRNA #3, BRCA1, or CDK12 siRNAs as described above. The cells were then plated in 24-well plates in RPMI-1640 supplemented with 10% fetal bovine serum with antibiotics, cultured for 48 hours, and analyzed for ATP content using a colorimetric ATP assay (Cat. No. ab83355, Abcam) and BRCA1 and CDK12 mRNA levels by qRT-PCR.

### Correlation Analyses of BRCA1 and CDK12 Versus NNMT in Patient and PDX Tumors

CDK12, BRCA1, and NNMT mRNA levels were obtained from an expression analysis in 98 HGSOC patient tumors and 127 non-overlapping HGSOC PDX models grown in mice. To assess the correlation between NNMT mRNA levels with CDK12 and BRCA1, Spearman correlation analysis was performed.

### Example 10: Exemplary Embodiments

Embodiment 1. A method for treating a mammal having cancer, wherein said method comprises:

(a) identifying said mammal as having a BRCA1-deficient cancer, and

(b) administering an oxidative phosphorylation (OXPHOS) inhibitor to said mammal to increase the susceptibility of said cancer to treatment with a platinum compound or a poly(ADP-ribose) polymerase (PARP) inhibitor.

Embodiment 2. The method of Embodiment 1, wherein said mammal is a human.
Embodiment 3. The method of any one of Embodiments 1-2, wherein said BRCA1-deficient cancer is selected from the group consisting of ovarian cancers, breast cancers, pancreatic cancers, and prostate cancers.
Embodiment 4. The method of Embodiment 3, wherein said BRCA1-deficient cancer is an ovarian cancer.
Embodiment 5. The method of any one of Embodiments 1-4, wherein said OXPHOS inhibitor is selected from the group consisting of VLX600 and ceritinib.
Embodiment 6. The method of Embodiment 5, wherein said OXPHOS inhibitor is VLX600.
Embodiment 7. The method of any one of Embodiments 1-6, said method further comprising administering said platinum compound to said mammal.
Embodiment 8. The method of Embodiment 7, wherein said platinum compound is cisplatin.
Embodiment 9. The method of any one of Embodiments 1-8, said method further comprising administering said PARP inhibitor to said mammal.
Embodiment 10. The method of Embodiment 9, wherein said PARP inhibitor is olaparib.
Embodiment 11. A method for treating a mammal having cancer, wherein said method comprises administering an oxidative phosphorylation (OXPHOS) inhibitor to a mammal identified as having a BRCA1-deficient cancer under conditions wherein the susceptibility of said cancer to treatment with a platinum compound or a poly(ADP-ribose) polymerase (PARP) inhibitor increases.
Embodiment 12. The method of Embodiment 11, wherein said mammal is a human.
Embodiment 13. The method of any one of Embodiments 11-12, wherein said BRCA1-deficient cancer is selected from the group consisting of ovarian cancers, breast cancers, pancreatic cancers, and prostate cancers.
Embodiment 14. The method of Embodiment 13, wherein said BRCA1-deficient cancer is an ovarian cancer.
Embodiment 15. The method of any one of Embodiments 11-14, wherein said OXPHOS inhibitor is selected from the group consisting of VLX600 and ceritinib.
Embodiment 16. The method of Embodiment 15, wherein said OXPHOS inhibitor is VLX600.
Embodiment 17. The method of any one of Embodiments 11-16, said method further comprising administering said platinum compound to said mammal.
Embodiment 18. The method of Embodiment 17, wherein said platinum compound is cisplatin.
Embodiment 19. The method of any one of Embodiments 11-18, wherein said method further comprising administering said PARP inhibitor to said mammal.
Embodiment 20. The method of Embodiment 19, wherein said PARP inhibitor is olaparib.
Embodiment 21. A method for treating a mammal having cancer, wherein said method comprises:

(a) identifying said mammal as having a BRCA1-deficient cancer, and

(b) administering an inhibitor of a mitochondrial polypeptide to said mammal to increase the susceptibility of said cancer to treatment with a platinum compound or a poly(ADP-ribose) polymerase (PARP) inhibitor.

Embodiment 22. The method of Embodiment 21, wherein said mammal is a human.
Embodiment 23. The method of any one of Embodiments 21-22, wherein said BRCA1-deficient cancer is selected from the group consisting of ovarian cancers, breast cancers, pancreatic cancers, and prostate cancers.
Embodiment 24. The method of Embodiment 23, wherein said BRCA1-deficient cancer is an ovarian cancer.
Embodiment 25. The method of Embodiment 23, wherein said BRCA1-deficient cancer is a breast cancer.
Embodiment 26. The method of any one of Embodiments 21-25, wherein said inhibitor of a mitochondrial polypeptide is tigecycline.
Embodiment 27. The method of any one of Embodiments 21-26, said method further comprising administering said platinum compound to said mammal.
Embodiment 28. The method of Embodiment 27, wherein said platinum compound is cisplatin.
Embodiment 29. The method of any one of Embodiments 21-28, said method further comprising administering said PARP inhibitor to said mammal.
Embodiment 30. The method of Embodiment 29, wherein said PARP inhibitor is olaparib.
Embodiment 31. A method for treating a mammal having cancer, wherein said method comprises administering an inhibitor of a mitochondrial polypeptide to a mammal identified as having a BRCA1-deficient cancer under conditions wherein the susceptibility of said cancer to treatment with a platinum compound or a poly(ADP-ribose) polymerase (PARP) inhibitor increases.
Embodiment 32. The method of Embodiment 31, wherein said mammal is a human.
Embodiment 33. The method of any one of claims 31-32, wherein said BRCA1-deficient cancer is selected from the group consisting of ovarian cancers, breast cancers, pancreatic cancers, and prostate cancers.
Embodiment 34. The method of Embodiment 33, wherein said BRCA1-deficient cancer is an ovarian cancer.
Embodiment 35. The method of Embodiment 33, wherein said BRCA1-deficient cancer is a breast cancer.
Embodiment 36. The method of any one of Embodiments 31-35, wherein said inhibitor of a mitochondrial polypeptide is tigecycline.
Embodiment 37. The method of any one of Embodiments 31-36, said method further comprising administering said platinum compound to said mammal.
Embodiment 38. The method of Embodiment 37, wherein said platinum compound is cisplatin.
Embodiment 39. The method of any one of Embodiments 31-38, said method further comprising administering said PARP inhibitor to said mammal.
Embodiment 40. The method of Embodiment 39, wherein said PARP inhibitor is olaparib.
Embodiment 41. A method for treating a mammal having cancer, wherein said method comprises:

(a) identifying said mammal as having a BRCA1-deficient cancer, and

(b) administering an inhibitor of glucose transport to said mammal to increase the susceptibility of said cancer to treatment with a platinum compound or a poly(ADP-ribose) polymerase (PARP) inhibitor.

Embodiment 42. The method of Embodiment 41, wherein said mammal is a human.
Embodiment 43. The method of any one of Embodiments 41-42, wherein said BRCA1-deficient cancer is selected from the group consisting of ovarian cancers, breast cancers, pancreatic cancers, and prostate cancers.
Embodiment 44. The method of Embodiment 43, wherein said BRCA1-deficient cancer is an ovarian cancer.
Embodiment 45. The method of any one of Embodiments 41-44, wherein said inhibitor of glucose transport can reduce or eliminate the expression and/or activity of a glucose transporter 1 (GLUT1) polypeptide.
Embodiment 46. The method of any one of Embodiments 41-45, wherein said inhibitor of glucose transport is WZB117.
Embodiment 47. The method of any one of Embodiments 41-46, said method further comprising administering said platinum compound to said mammal.
Embodiment 48. The method of Embodiment 47, wherein said platinum compound is cisplatin.
Embodiment 49. The method of any one of Embodiments 41-48, said method further comprising administering said PARP inhibitor to said mammal.
Embodiment 50. The method of Embodiment 49, wherein said PARP inhibitor is olaparib.
Embodiment 51. A method for treating a mammal having cancer, wherein said method comprises administering an inhibitor of glucose transport to a mammal identified as having a BRCA1-deficient cancer under conditions wherein the susceptibility of said cancer to treatment with a platinum compound or a poly(ADP-ribose) polymerase (PARP) inhibitor increases.
Embodiment 52. The method of Embodiment 51, wherein said mammal is a human.
Embodiment 53. The method of any one of Embodiments 51-52, wherein said BRCA1-deficient cancer is selected from the group consisting of ovarian cancers, breast cancers, pancreatic cancers, and prostate cancers.
Embodiment 54. The method of Embodiment 53, wherein said BRCA1-deficient cancer is an ovarian cancer.
Embodiment 55. The method of any one of Embodiments 51-54, wherein said inhibitor of glucose transport can reduce or eliminate the expression and/or activity of a GLUT1 polypeptide.
Embodiment 56. The method of Embodiment 55, wherein said inhibitor of glucose transport is WZB117.
Embodiment 57. The method of any one of Embodiments 51-56, said method further comprising administering said platinum compound to said mammal.
Embodiment 58. The method of Embodiment 57, wherein said platinum compound is cisplatin.
Embodiment 59. The method of any one of Embodiments 51-58, said method further comprising administering said PARP inhibitor to said mammal.
Embodiment 60. The method of Embodiment 59, wherein said PARP inhibitor is olaparib.
Embodiment 61. A method for treating a mammal having cancer, wherein said method comprises:

(a) identifying said mammal as having a nicotinamide N-methyltransferase (NNMT) overexpressing cancer, and

(b) administering an oxidative phosphorylation (OXPHOS) inhibitor to said mammal to increase the susceptibility of said cancer to treatment with a platinum compound or a poly(ADP-ribose) polymerase (PARP) inhibitor.

Embodiment 62. The method of Embodiment 61, wherein said mammal is a human.
Embodiment 63. The method of any one of Embodiments 61-62, wherein said NNMT overexpressing cancer is selected from the group consisting of ovarian cancers, breast cancers, pancreatic cancers, and prostate cancers.
Embodiment 64. The method of Embodiment 63, wherein said NNMT overexpressing cancer is an ovarian cancer.
Embodiment 65. The method of any one of Embodiments 61-64, wherein said OXPHOS inhibitor is selected from the group consisting of VLX600 and ceritinib.
Embodiment 66. The method of Embodiment 65, wherein said OXPHOS inhibitor is VLX600.
Embodiment 67. The method of any one of Embodiments 61-66, said method further comprising administering said platinum compound to said mammal.
Embodiment 68. The method of Embodiment 67, wherein said platinum compound is cisplatin.
Embodiment 69. The method of any one of Embodiments 61-68, said method further comprising administering said PARP inhibitor to said mammal.
Embodiment 70. The method of Embodiment 69, wherein said PARP inhibitor is olaparib.
Embodiment 71. A method for treating a mammal having cancer, wherein said method comprises administering an oxidative phosphorylation (OXPHOS) inhibitor to a mammal identified as having a nicotinamide N-methyltransferase (NNMT) overexpressing cancer under conditions wherein the susceptibility of said cancer to treatment with a platinum compound or a poly(ADP-ribose) polymerase (PARP) inhibitor increases.
Embodiment 72. The method of Embodiment 71, wherein said mammal is a human.
Embodiment 73. The method of any one of Embodiments 71-72, wherein said NNMT overexpressing cancer is selected from the group consisting of ovarian cancers, breast cancers, pancreatic cancers, and prostate cancers.
Embodiment 74. The method of Embodiment 73, wherein said NNMT overexpressing cancer is an ovarian cancer.
Embodiment 75. The method of any one of Embodiments 71-74, wherein said OXPHOS inhibitor is selected from the group consisting of VLX600 and ceritinib.
Embodiment 76. The method of Embodiment 75, wherein said OXPHOS inhibitor is VLX600.
Embodiment 77. The method of any one of Embodiments 71-76, said method further comprising administering said platinum compound to said mammal.
Embodiment 78. The method of Embodiment 77, wherein said platinum compound is cisplatin.
Embodiment 79. The method of any one of Embodiments 71-78, said method further comprising administering said PARP inhibitor to said mammal.
Embodiment 80. The method of Embodiment 79, wherein said PARP inhibitor is olaparib.
Embodiment 81. A method for treating a mammal having cancer, wherein said method comprises:

(a) identifying said mammal as having a nicotinamide N-methyltransferase (NNMT) overexpressing cancer, and

(b) administering an inhibitor of a mitochondrial polypeptide to said mammal to increase the susceptibility of said cancer to treatment with a platinum compound or a poly(ADP-ribose) polymerase (PARP) inhibitor.

Embodiment 82. The method of Embodiment 81, wherein said mammal is a human.
Embodiment 83. The method of any one of Embodiments 81-82, wherein said NNMT overexpressing cancer is selected from the group consisting of ovarian cancers, breast cancers, pancreatic cancers, and prostate cancers.
Embodiment 84. The method of Embodiment 83, wherein said NNMT overexpressing cancer is an ovarian cancer.
Embodiment 85. The method of Embodiment 83, wherein said NNMT overexpressing cancer is a breast cancer.
Embodiment 86. The method of any one of Embodiments 81-85, wherein said inhibitor of a mitochondrial polypeptide is tigecycline.
Embodiment 87. The method of any one of Embodiments 81-86, said method further comprising administering said platinum compound to said mammal.
Embodiment 88. The method of Embodiment 87, wherein said platinum compound is cisplatin.
Embodiment 89. The method of any one of Embodiments 81-88, said method further comprising administering said PARP inhibitor to said mammal.
Embodiment 90. The method of Embodiment 89, wherein said PARP inhibitor is olaparib.
Embodiment 91. A method for treating a mammal having cancer, wherein said method comprises administering an inhibitor of a mitochondrial polypeptide to a mammal identified as having a nicotinamide N-methyltransferase (NNMT) overexpressing cancer under conditions wherein the susceptibility of said cancer to treatment with a platinum compound or a poly(ADP-ribose) polymerase (PARP) inhibitor increases.
Embodiment 92. The method of Embodiment 91, wherein said mammal is a human.
Embodiment 93. The method of any one of Embodiments 91-92, wherein said NNMT overexpressing cancer is selected from the group consisting of ovarian cancers, breast cancers, pancreatic cancers, and prostate cancers.
Embodiment 94. The method of Embodiment 93, wherein said NNMT overexpressing cancer is an ovarian cancer.
Embodiment 95. The method of Embodiment 93, wherein said NNMT overexpressing cancer is a breast cancer.
Embodiment 96. The method of any one of Embodiments 91-95, wherein said inhibitor of a mitochondrial polypeptide is tigecycline.
Embodiment 97. The method of any one of Embodiments 91-96, said method further comprising administering said platinum compound to said mammal.
Embodiment 98. The method of Embodiment 97, wherein said platinum compound is cisplatin.
Embodiment 99. The method of any one of Embodiments 91-98, said method further comprising administering said PARP inhibitor to said mammal.
Embodiment 100. The method of Embodiment 99, wherein said PARP inhibitor is olaparib.
Embodiment 101. A method for treating a mammal having cancer, wherein said method comprises:

(a) identifying said mammal as having a nicotinamide N-methyltransferase (NNMT) overexpressing cancer, and

(b) administering an inhibitor of glucose transport to said mammal to increase the susceptibility of said cancer to treatment with a platinum compound or a poly(ADP-ribose) polymerase (PARP) inhibitor.

Embodiment 102. The method of Embodiment 101, wherein said mammal is a human.
Embodiment 103. The method of any one of claims 101-102, wherein said NNMT overexpressing cancer is selected from the group consisting of ovarian cancers, breast cancers, pancreatic cancers, and prostate cancers.
Embodiment 104. The method of Embodiment 103, wherein said NNMT overexpressing cancer is an ovarian cancer.
Embodiment 105. The method of any one of Embodiments 101-104, wherein said inhibitor of glucose transport can reduce or eliminate the expression and/or activity of a glucose transporter 1 (GLUT1) polypeptide.
Embodiment 106. The method of any one of Embodiments 101-105, wherein said inhibitor of glucose transport is WZB117.
Embodiment 107. The method of any one of Embodiments 101-106, said method further comprising administering said platinum compound to said mammal.
Embodiment 108. The method of Embodiment 107, wherein said platinum compound is cisplatin.
Embodiment 109. The method of any one of Embodiments 101-108, said method further comprising administering said PARP inhibitor to said mammal.
Embodiment 110. The method of Embodiment 109, wherein said PARP inhibitor is olaparib.
Embodiment 111. A method for treating a mammal having cancer, wherein said method comprises administering an inhibitor of glucose transport to a mammal identified as having a nicotinamide N-methyltransferase (NNMT) overexpressing cancer under conditions wherein the susceptibility of said cancer to treatment with a platinum compound or a poly(ADP-ribose) polymerase (PARP) inhibitor increases.
Embodiment 112. The method of Embodiment 111, wherein said mammal is a human.
Embodiment 113. The method of any one of Embodiments 111-112, wherein said NNMT overexpressing cancer is selected from the group consisting of ovarian cancers, breast cancers, pancreatic cancers, and prostate cancers.
Embodiment 114. The method of Embodiment 113, wherein said NNMT overexpressing cancer is an ovarian cancer.
Embodiment 115. The method of any one of Embodiments 111-114, wherein said inhibitor of glucose transport can reduce or eliminate the expression and/or activity of a GLUT1 polypeptide.
Embodiment 116. The method of Embodiment 115, wherein said inhibitor of glucose transport is WZB117.
Embodiment 117. The method of any one of Embodiments 111-116, said method further comprising administering said platinum compound to said mammal.
Embodiment 118. The method of Embodiment 117, wherein said platinum compound is cisplatin.
Embodiment 119. The method of any one of Embodiments 111-118, said method further comprising administering said PARP inhibitor to said mammal.
Embodiment 120. The method of Embodiment 119, wherein said PARP inhibitor is olaparib.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having cancer, wherein said method comprises administering an oxidative phosphorylation (OXPHOS) inhibitor to a mammal identified as having a BRCA1-deficient cancer under conditions wherein the susceptibility of said cancer to treatment with a platinum compound or a poly(ADP-ribose) polymerase (PARP) inhibitor increases.

2. The method of claim 1, wherein said mammal is a human.

3. The method of claim 1, wherein said BRCA1-deficient cancer is selected from the group consisting of ovarian cancers, breast cancers, pancreatic cancers, prostate cancers, skin cancers, renal cancers, liver cancers, stomach cancers, colon cancers, colorectal cancers, bladder cancers, and oral squamous cell cancers.

4. The method of claim 1, wherein said OXPHOS inhibitor is selected from the group consisting of VLX600 and ceritinib.

5. The method of claim 1, said method further comprising administering said platinum compound to said mammal.

6. (canceled)

7. The method of claim 1, said method further comprising administering said PARP inhibitor to said mammal.

8. (canceled)

9. A method for treating a mammal having cancer, wherein said method comprises administering an inhibitor of a mitochondrial polypeptide to a mammal identified as having a BRCA1-deficient cancer under conditions wherein the susceptibility of said cancer to treatment with a platinum compound or a poly(ADP-ribose) polymerase (PARP) inhibitor increases.

10. The method of claim 9, wherein said mammal is a human.

11. The method of claim 9, wherein said BRCA1-deficient cancer is selected from the group consisting of ovarian cancers, breast cancers, pancreatic cancers, prostate cancers, skin cancers, renal cancers, liver cancers, stomach cancers, colon cancers, colorectal cancers, bladder cancers, and oral squamous cell cancers.

12. The method of claim 9, wherein said inhibitor of a mitochondrial polypeptide is tigecycline.

13. The method of claim 9, said method further comprising administering said platinum compound to said mammal.

14. (canceled)

15. The method of claim 9, said method further comprising administering said PARP inhibitor to said mammal.

16. (canceled)

17. A method for treating a mammal having cancer, wherein said method comprises administering an inhibitor of glucose transport to a mammal identified as having a BRCA1-deficient cancer under conditions wherein the susceptibility of said cancer to treatment with a platinum compound or a poly(ADP-ribose) polymerase (PARP) inhibitor increases.

18. The method of claim 17, wherein said mammal is a human.

19. The method of claim 17, wherein said BRCA1-deficient cancer is selected from the group consisting of ovarian cancers, breast cancers, pancreatic cancers, prostate cancers, skin cancers, renal cancers, liver cancers, stomach cancers, colon cancers, colorectal cancers, bladder cancers, and oral squamous cell cancers.

20. The method of claim 17, wherein said inhibitor of glucose transport can reduce or eliminate the expression and/or activity of a GLUT1 polypeptide.

21. The method of claim 20, wherein said inhibitor of glucose transport is WZB117.

22. The method of claim 17, said method further comprising administering said platinum compound to said mammal.

23. (canceled)

24. The method of claim 17, said method further comprising administering said PARP inhibitor to said mammal.

25. (canceled)
